(19) United States
(12) Patent Application Publication
(10) Pub. No.: US 2007/0232885 A1
(43) Pub. Date: Oct. 4, 2007

(54) Medical imaging examination review and quality assurance system and method

(76) Inventors: Thomas Cook, Lexington, SC (US); Robert E. Hylas, Allendale, NJ (US); Patrick Hunt, Columbia, SC (US)

Correspondence Address: TRIANGLE PATENTS, P.L.L.C., P.O. BOX 28539, RALEIGH, NC, 27611-8539, US

(21) Appl. No.: 11/713,345

(22) Filed: Mar. 2, 2007

Related U.S. Application Data
(60) Provisional application No. 60/779,332, filed on Mar. 3, 2006.

(51) Int. Cl. A61B 5/05

(52) U.S. Cl. 600/407

(57) ABSTRACT

The present invention is a computer-based review and quality assurance system and method for physicians who use portable imaging technology to diagnose and treat patients at a point of care. The system and method facilitates the credentialing of physicians, provides on-going quality assurance (QA), and integrates medical imaging equipment with software that runs over a network. Such credentialing assists medical institutions by identifying physicians who effectively use and diagnose patients with the imaging equipment.

### CROSS-REFERENCE TO RELATED APPLICATIONS

This non-provisional utility patent application claims the benefit of provisional application Ser. No. 60/779,332, filed Mar. 3, 2006, which is incorporated herein by reference in its entirety.

### BACKGROUND OF THE INVENTION

(1) Field of the Invention

The present invention relates generally to credentialing physicians and, more particularly, to a computer-based quality assurance system for physicians who use portable imaging technology to diagnose and treat patients at a point of care.

(2) Description of the Prior Art

With digitization and increased miniaturization of electronic equipment, physicians today are beginning to be able to use portable medical imaging devices at the initial point of care to directly diagnose patients. This practice, when performed by properly trained and credentialed physicians, holds great promise to both reduce medical treatment costs and save lives. We may be at the dawn of a new age in medicine where imaging devices such as ultrasound will become in effect the digital stethoscope of the 21st century.

Despite these breakthroughs, physicians who wish to receive the training and become credentialed to use new portable medical imaging devices at the initial point of care face formidable barriers. Specifically, it is extremely difficult for qualified physicians to receive the type of training and feedback they need to become credentialed or in some other way officially recognized as qualified to use portable medical imaging technologies at the point of care. This is true even in cases where there are clear national guidelines and local requirements in place for how a physician should become credentialed to use medical imaging based upon well established standards.

There are a plethora of devices today used in medicine to capture internal images of the human body that can be used to diagnose patients. These include, for example, X-ray, computer tomography (“CT” or previously know as CAT scan), magnetic resonance imaging (“MRI”), ultrasound (“US”), computer radiography (“CR”), mammography, and nuclear medicine (“NM”). These different pieces of equipment are commonly known as “modalities” in the healthcare and medical equipment industry.

Traditionally, the use of medical imaging technology has been almost the exclusive province of specialized medical technicians who use the equipment to capture images of patients and specialized physicians known as radiologists who interpret the images captured by these technicians. Originally, due to its sheer size and complexity, medical imaging equipment was typically located in a single department or separate group offering the services to multiple users and was for the most part not mobile.

This is changing. In recent years, one imaging modality in particular, ultrasound has become much more portable. This includes recent introductions of both handheld and laptop sized ultrasound devices. Other medical imaging technologies such as CT scan are also being miniaturized like ultrasound for use directly at the initial point of care. Other modalities are expected to follow this trend towards miniaturization in the years to come.

With this innovation, physicians are beginning to realize in certain instances they can use handheld or other highly mobile imaging devices directly to diagnose patients rather than solely relying on radiology departments or groups to use the equipment on their behalf. This practice is not always appropriate. In fact, a physician needs to determine when to use medical imaging technology themselves versus when to rely on the radiologist. As such, the practice requires additional training and development of altogether new types of skills on the part of the physicians to use imaging equipment themselves.

Today multiple specialties are beginning to utilize ultrasound in the clinical arena. For example, surgeons are using ultrasound to evaluate and treat patients in several areas, including trauma, thyroid and breast ultrasound, as well as line placement and other procedures. Internal medicine and family practice physicians utilize ultrasound for screening of abdominal aortic aneurism's (AAA's) and gallbladder disease. Critical Care physicians use ultrasound to evaluate pulmonary status and cardiac function in ICU (intensive care units). Nephrologists use ultrasound to assist in catheter placement as well.

Emergency medicine in particular is the medical specialty area where physicians are most rapidly adopting ultrasound as a direct diagnostic tool. However, as indicated above other specialties are starting to follow suite including internal medicine, trauma surgeons, family practice physicians, etc. often using practices first established by emergency physicians.

Research indicates that for certain medical conditions, use of a portable imaging device immediately by a qualified emergency or other physician can save lives. Certain life threatening conditions can be detected more quickly with ultrasound imaging than would otherwise be possible if the physician had to wait to schedule an imaging exam through another group.

To demonstrate competence, however, physicians who wish to use portable or other medical imaging devices should be credentialed to use the technology. As an example, emergency physicians can become credentialed to use ultrasound at the point of care by meeting certain minimum requirements established by their hospital or health institution.

Although credentialing requirements are established by a local credentialing body, hospitals and health institutions generally rely on guidelines for credentialing provided to them by national physician organizations, occasionally with only minor modifications to meet local requirements or preferences. For emergency physicians, guidelines on the use of ultrasound are established by the American College of Emergency Physicians (ACEP). These guidelines are a good example of how physicians are credentialed to use portable medical imaging devices; however the respective societies of medical specialties will decide the appropriate number of studies for their physicians.

In addition to classroom training, the ACEP guidelines require emergency physicians to perform anywhere from 150 to 300 ultrasound examinations for training purposes on actual patients. These include examinations conducted across 6 different examination (“exam”) types specified in the guidelines including OB-GYN, aorta, gall bladder, kidney, cardiac and trauma. Additionally, to become credentialed, the guidelines require the physician to obtain feedback on each of these 150 to 300 exams preformed from a qualified physician or other medical professional who is already credentialed in the use of the imaging technology.

ACEP guidelines are reasonably typical of the types of training and feedback required of physicians to become credentialed or otherwise officially recognized to use portable imaging devices. These types of guidelines are modeled on the assumption that timely feedback can be provided not unlike those processes used in the Middle Ages for Guilds. That is the process assumes that an already qualified physician will literally look over the shoulder of another physician using the equipment to perform a medical examination to immediately provide feedback. Unfortunately, as technology advanced, particularly medical imaging technology, the necessary skills to train and credential physicians to use the technologies are initially quite scarce, severely limiting the ability to provide this type of feedback and likewise severely limiting the speed of adoption. For portable ultrasound used in emergency situations this slow adoption process more than likely means that lives will be lost due to antiquated methods for training and credentialing physicians to use the technology.

With the emergence of handheld and other portable medical imaging devices, physicians are attempting to receive the types of direct educational feedback and on-going quality assurance needed for credentialing to use the technology to practice medicine. Unfortunately, receiving this type of feedback is difficult as the type of feedback they need is unique and, in many cases, individuals qualified to supply the feedback are not available locally.

These difficulties are due to a number of challenges that restrict the process. Today, physicians who are seeking the type of feedback and review needed to become credentialed to use, for instance, portable ultrasound, must for the most part resort to manual means. Today, these means include one of two methods as described below.

The most common method requires printing out of paper or thermal copies of, e.g., ultrasound, images and providing these to reviewers for feedback. Typically, these print outs would have to be pasted onto a piece of paper so that they would not be lost, with sometimes more than one image per page.

Next, the physician manually completes a paper-based worksheet or some exam form to document his or her findings for the six different ACEP proscribed medical exam types. The physician must find a qualified physician or other clinical professional who can provide written feedback on the exams. Both the images and written findings then have to be forwarded to the reviewer usually by mail.

After the review is completed, the physician needs to wait for the feedback to be returned and review the feedback provided on the images and the findings from the reviewer. The physician or an administrative support person compiles statistics on progress and keeps track of how many exams by type of exam the physician has received feedback on including compiling statistics on progress and on how many for each exam type have yet to be completed.

Upon completion of the process, the physician or an administrative support person must document the full results of the “overread” exams or reviews, and provide these to the institution as evidence that the physician has completed the credentialing requirements for that particular institution.

Needless to say, this is a very cumbersome process.

Another method commonly used is to make video tapes of ultrasound exams and to provide these to reviewers in remote locations. This approach has some advantages over the use of thermal printouts as motion videos or so called video “clips” can be provided to the reviewer.

However, this process is also quite cumbersome as the physician needs to ensure their own videos are those included on the tape. Frequently in emergency departments several physicians might record ultrasound videos using the same tape run using a video machine that the vendors commonly mount on mobile carts designed specifically for the mobile imaging machine.

Additionally, even when a physician is able to exclude everyone else's videos from the tape, the physician must also indicate to the reviewer which video clips or still pictures are associated with which exam write-up. The physician again has to provide the video tape to the reviewer again typically by mail. The physician has to again wait for the feedback either by mail or via e-mail from the reviewer and associate the specific feedback with the right medical images on the tape.

Also, the physician or administrative support staff has to keep track of how many exams have been completed. Finally, the physician has to complete and receive feedback on anywhere from 150 to 300 exams to become credentialed depending upon the institution.

As a result of the above situation, research confirms a significant percentage of “phantom” imaging exams are performed today by uncredentialed emergency physicians. A phantom exam occur when, for example, an emergency physician who has in fact received some level of classroom training to use mobile imaging technology does not report usage of the equipment to examine a patient because he or she is not yet officially credentialed to use the equipment.

The physician may in fact have achieved some level of competence to use the equipment but has not yet been credentialed because of the time consuming nature and complexity of the processes described above. Particularly with life threatening situations, the physician apparently chooses to perform a phantom exam by using the equipment without being fully credentialed, rather than to risk missing a potentially time sensitive life threatening condition by waiting to schedule an imaging study with radiology.

This research indicates that anywhere from 70-80% of the exams performed by emergency physicians using ultrasound are not currently reported. Although it is in some ways admirable that these physicians are using the technology because they believe they might need to save lives, it is not a good situation that these physicians have not been able to take the time and find the means to also become officially credentialed to use the technology.

A major reason for this situation is that there is the lack of any efficient system and method for physicians to become credentialed. Likewise, once physicians are credentialed to use medical imaging devices there is no efficient system or method for monitoring their continued competence to use the technology through some type of periodic quality assurance (“QA”) process. One might expect that there would be systems and technical infrastructures in place to support this type of process. However, hospital systems today do not support to any great extent or through a comprehensive process the types of secure sharing of medical information across multiple different organizations or directly between physicians that is required to support electronic credentialing of physicians to use mobile ultrasound devices as described above let alone offering a specific application to support the process.

Today, medical images generated within hospitals and other clinical settings are typically stored in systems referred to as Picture Archive and Communications Systems (PACS).

Originally, medical imaging technologies such as x-ray involved the generation of film used to process and display images collected by the equipment. Today, medical imaging film is being rapidly replaced by digital technologies. As such, film is rapidly being replaced by digital technology for medical imaging. A large percentage of medical imaging equipment, though, still uses film-based storage medium. Further, many hospitals still store a great deal of old film-based medical images.

Historically, medical imaging equipment used film to capture medical images. In recent years, the shift from film-based to digital medical imaging is part of the reason why this equipment can be more easily miniaturized. Most, if not all, portable imaging devices appear to use digital images in lieu of film to capture and record the images.

Film creates well known challenges for sharing and transporting medical images. However, digital images are much easier to share across a network throughout a hospital or even around the world using a network given proper security. However, with the current technology, there is not a satisfactory procedure for physicians to take advantage of digital images taken with portable imaging equipment.

As with any major advance in technology, the introduction of portable medical imaging, despite its benefits, creates altogether new adoption challenges yet to be addressed. The primary challenge is training and credentialing of physicians to use the equipment under appropriate circumstances at the initial point of care.

Due to the change from the traditional applications of medical imaging, there is a corresponding change in the areas where a physician requires competency. This change is the reason for the required credentialing discussed above. In contrast to traditional applications of medical imaging, a portable medical imaging device used by physicians requires the physician to become competent in both a) the use of the equipment and b) the interpretation of the findings. Traditionally, these two functions have been separate with one individual trained to use the equipment (i.e., technologists or “sonographers” for ultrasound) and another trained to interpret the images (i.e., a physician known as a radiologist).

Traditionally medical imaging equipment was too large and bulky to normally bring to a first point-of-patient medical care to diagnose a patient and too difficult to operate for anyone other than a trained technologist. Instead, much like mainframe technology in the field of information technology (“IT”), medical imaging in healthcare was managed as a shared resource and support service group. To use medical imaging, physicians traditionally relied on specialized radiology departments or radiology groups to acquire and interpret medical images for them rather than use the equipment themselves.

However, in recent years it has become more and more possible, through digitization of images and miniaturization of the devices, for physicians themselves in some cases (not all) to use the equipment immediately at the first point of care.

To date, ultrasound technology has been the most predominant imaging modality that has been miniaturized to a point where physicians can use it at the point of care. As mentioned earlier, further miniaturization of ultrasound as well as other imaging modalities is expected to continue (e.g., CT scan, MRI, etc.) in the future.

However, how a non-radiology physician uses a medical imaging device is very different from how either the radiologist or a technician separately would use the device. Radiologists generally rely on the medical technicians to use the device to acquire images. Technicians are trained and competent to use the equipment to acquire images but not qualified to interpret the images. Neither the technician nor the radiologist is 100% aware of everything the physician who originally treated and diagnosed the patient knows.

In contrast, physicians who wish to use portable imaging themselves must learn both how to use the device and how to interpret the images immediately. Although this is a challenge, as the physician has to become competent in two disciplines that the physician previously did not have to be competent in, there are significant advantages.

The most obvious advantage is speed. With portable medical imaging, a physician can more quickly diagnose a patient. This is particularly important when there is a life threatening situation where time is of the essence. There are many cases of life threatening conditions that if left undiagnosed for any period of time where the patient will otherwise die that cannot be diagnosed without use of the specific medical imaging technology.

A good example is bleeding in the abdomen. When a patient has an accident such as a fall the patient may or may not have a life threatening condition. One condition that could in fact be life threatening but is relatively painless is internal bleeding in the abdomen. The use of ultrasound can quickly detect whether there is internal bleeding.

But delays in scanning a patient with ultrasound could mean the difference between life and death. The treatment for a ruptured abdominal aortic aneurism is emergency surgery to stop the internal bleeding. But without immediate access to a mobile imaging device the patient might not be treated soon enough to save his or her life.

Another example is an ectopic pregnancy. A pregnant woman who is experiencing some level of discomfort could be experiencing any one of a number of different conditions often associated with pregnancy most of which are not life threatening to the mother and the fetus. However, ectopic pregnancy is one condition that is clearly life threatening when symptoms commence and could result in death if not treated very quickly once the symptoms commence.

It is very difficult to distinguish the symptoms of an ectopic pregnancy from many other causes of discomfort when a woman is pregnant. One highly reliable way, however, to determine if a pregnant woman has an ectopic pregnancy is through the use of ultrasound. But here again time is critical.

So providing physicians with a method that allows them to more readily become credentialed to use medical imaging devices is important. Additionally, it can reduce the cost of healthcare by speeding the diagnosis and treatment of patients. But most important it can save lives by ensuring that physicians can readily become credentialed to use these important advances in medical technology.

As such, what is needed is an invention geared towards the unique needs of physicians who wish to become competent to use medical imaging technology at the initial point of medical care.

Radiologists and technicians already have well established training and certification programs to separately qualify them to use medical imaging equipment in their own specified roles. However, the processes and programs for medical imaging technologists and radiologists to become competent to use medical imaging technology are very different from those of other specialties and do not satisfy the needs of these physicians who wish to use portable medical imaging themselves.

Physicians use ultrasound differently than sonographers and radiologists. For example, emergency physicians use ultrasound to conduct “focused” exams with an objective of quickly identifying whether or not certain life threatening conditions are present (i.e., answer a “yes” or “no” question). Examples include an ectopic pregnancy, traumatic hemoperitoneum, abdominal aortic aneurism, cardiac tamponade, pneumothorax, etc.

In contrast, technologists and radiologists are called upon to conduct more “comprehensive” examinations. These encompass a much more thorough but also time consuming diagnosis of a particular area of the body and documentation of clinical findings by writing a thorough narrative report, as opposed to an emergency physician's quick focus primarily on life threatening conditions.

With greater miniaturization of medical imaging technology, it is more feasible to deploy imaging technology directly at the initial point of care. It is not just the point of care physicians who benefit from miniaturization of this technology. In some cases, miniaturized imaging devices allow radiology technicians to become more mobile and available to immediately work with physicians at the initial point of medical care to acquire images and quickly transmit these to radiology departments for rapid diagnosis.

The above is a good development, but is not adequate for training and credentialing a physician. The present invention is concerned with unique methods and techniques needed to train and credential a physician to directly use portable medical imaging.

The American Medical Association (“AMA”) in the US has recognized the need to address standards for when physicians choose to use medical imaging immediately at the first point of care. The AMA established a policy that the specialty medical societies themselves should determine when it is and is not appropriate for a physician in a given specialty to use an imaging device, such as ultrasound, or otherwise rely on radiologist and ultrasound technicians. Additionally, the AMA policy charges each specialty medical society with establishing appropriate standards for credentialing or otherwise officially recognizing physicians in that specialty to directly use medical imaging devices such as is the case for the ACEP.

As a result, the medical profession in the US is gradually establishing guidelines and standards for certain non-radiology physician specialties to use medical imaging. Emergency medicine is a specialty that has in particular led in the usage of portable medical imaging using ultrasound at the first point of care. For example, the American College of Emergency Physicians (“ACEP”) has established clear guidelines for an emergency physician to use ultrasound.

To summarize, there are two distinct types of activities that are traditionally separated but lately are becoming combined when physicians directly use medical imaging devices at the initial point of care. These activities include:
A. the use of medical imaging equipment, and
B. the interpretation and evaluation of images to make diagnosis.

Traditionally, item A above has been the province of radiology technicians who use the equipment but are not trained or qualified to interpret the images. Traditionally item B above has been the province of specialized physicians or radiologists who are trained to interpret the images collected by technicians but not to use the equipment or acquire the images. The traditional procedure is illustrated in FIG. 1.

FIG. 2 presents a representation of a mobile imaging device for ultrasound. Ultrasound is by far the most prevalent example today of a medical imaging modality that has been significantly miniaturized such that physicians can now readily and productively use the technology to directly diagnose patients at an initial point of care. However, we are also seeing other medical imaging modalities becoming more mobile due to miniaturization such as mobile CT scanners. Therefore, the number of physicians who directly use mobile medical imaging devices to diagnose patients at an initial point of care is expected to increase and continue across a broad spectrum of medical specialties.

As illustrated in FIG. 3, due to the emergence of increasingly small medical imaging devices, physicians are starting to use the equipment themselves to immediately diagnose patients rather than solely relying on outside technicians and radiologists in every instance. When the process, including steps performed at a single location as shown in FIG. 3, is competently performed, great efficiencies over the process presented in FIG. 1, including four groups of steps performed at three locations, can be achieved. However, when activities A and B above are combined as presented in FIG. 1, the nature of the medical examination that can be performed by a physician becomes much different than other types of examinations performed in the past.

As an example, for emergency medicine, so called “focused” examinations can be performed quickly by a physician using ultrasound for each of several different specific types of medical conditions. The objective of a focused exam is to quickly check for specific potentially life threatening medical conditions and other serious medical conditions associated with the particular exam type (e.g., aorta, cardiac, OB-GYN, gall bladder, kidney, lung, trauma). If the right ultrasound images are collected, a focused exam lends itself to answering a short list of well established diagnostic questions with yes or no answers.

In contrast to emergency medicine, technicians and radiologists who use medical imaging equipment as pictured in FIG. 1 perform “comprehensive” exams. Not having actually conducted the initial examination with the patient, their objective is to cover all possibilities. As such, the technologist normally collects a complete sample of all possible images for a specified area of the body (e.g., abdomen) and the radiologist comprehensively analyzes the images acquired by the technician including preparing a comprehensive written report. In contrast, this process does not lend itself well to quickly answering a specific short list of well defined diagnostic questions with yes or no answers as is the case for general physician or specific emergencybased usage of ultrasound.

As such, the advent of mobile medical imaging devices creates a whole new type of medical examination that is already commonly performed in emergency medicine. Now it is also rapidly spreading to other medical specialties. The challenge, however, is for these physicians to secure the type of feedback necessary to be recognized as competent to perform these new types of exams. Particularly, they need to quickly and efficiently receive specific feedback to become credentialed by their local institutions. This enables them to completely and competently use various types of new mobile medical imaging devices and to maintain and improve these skills over time.

There are automated systems in hospitals used to store and process medical images. These systems are referred to as PACS systems. These systems are used to store and retrieve medical images taken from a variety of image modalities (e.g., ultrasound, x-ray, CT scan, etc.). However, as originally designed, the primary purpose of these PACS systems is to support the work processes and functions of radiology departments and radiology groups as depicted in FIG. 1; not to credential a physician.

Also, despite the existence of a standard called DICOM for uniformly structuring, transferring and storing medical images, there are nonetheless major differences in the file structures and image formats that different vendors use to physically download medical image files or groups of images grouped into “folders” onto external memory devices.

There is greater consistency, however, in how the vendors handle automated transfers of images and automated interfaces with hospital systems using DICOM standards. But this does not help the physician who today is manually printing out or, at best, downloading image folders for review and QA. Unfortunately, there is even more diversity in how vendors support non-automated physical downloading of images that can be executed by physicians and others directly. Differences can include, for instance, use of different image format standards such as bit maps versus J-peg images, and different structures used to set up image folders.

The DICOM standards do include standards for how medical image folders should be set up and transferred. However, these standards are not always followed and not uniformly interpreted the same way by different vendors.

Regardless, these are technical problems that a physician cannot and should not be called upon to address. Thus there is a need for a system that detects the vendor image file formats and loads the folders accordingly so as not to impede or prevent access to a broader review and QA process.

Collectively, there remains a need for an integrated system for training and certifying physicians to use and diagnose patients using portable medical imaging devices at the initial point of care.

### SUMMARY OF THE INVENTION

The present invention is directed to a computer-based review and quality assurance system for physicians who use portable imaging technology to diagnose and treat patients at a point of care. The system facilitates credentialing of the physicians and integrates medical imaging equipment with software that runs over a network. Such credentialing assists medical institutions by identifying physicians who can effectively use and diagnose patients with the imaging equipment.

The present invention is directed to a system and method that provides an automated procedure for credentialing physicians that is substantially less cumbersome than any conventional credentialing procedure. The procedure facilitates satisfaction of a strong demand for a physician to receive necessary training and credentialing to use portable imaging devices. It further provides for on-going QA.

In particular, due to well known healthcare industry security and privacy concerns and regulations, it is very difficult for a physician to actually transfer digital medical images without significant assistance from IT professionals. The present invention is directed to a system that aids in and simplifies the transfer of digital images, e.g., transferred as part of an exam for credentialing.

In the preferred embodiment, the imaging equipment is mobile and utilized at the point of care. The imaging equipment is preferably an ultrasound scanner, but may also include MRI, CT, and X-ray imaging modalities. The system provides simultaneous feedback regarding how to use the imaging equipment and how to interpret the images from a reviewer who may be remote to the physician being certified.

Preferably, the software of the present invention incorporates custom software for a variety of situations. Ideally, the software runs on an operating system, such as Microsoft Windows, and provides the following: universal encryption, workstation review for local and/or remote review of the physician's work, quality assurance functions, potential integration with an exchange service provider and remote review services, and administrative tasks. The software provides physicians with standardized exam templates that are completed and submitted by the physician. Copies of these templates contain the images that the physician acquires with the imaging equipment and his or her diagnostic findings. The templates review a physician's use of the imaging equipment and provide a new format for medical examinations or tests involved in the credentialing of a physician. Illustratively, these tests include the following categories: aorta, cardiac, gall bladder, kidney, lung, trauma, and obstetrics/gynecology.

The templates are preferably specific for each exam category and include specific focused questions directed towards the key medical findings and corresponding images from the imaging equipment. The templates collect information that can be used by a reviewer to determine the technical competence and accuracy of the physician's clinical findings based on the images that the physician has acquired. The templates allow the reviewer to determine whether the correct image views were acquired, whether the quality of the images is acceptable, and whether the overall image-based exam was acceptable. Lastly, the software can be integrated with educational electronic content, such as a Learning Management Portal (i.e., on-line learning systems) to supplement a physician's or a medical student's on-line learning experiences.

The software is enabled to run over a network and this is preferably the Internet. Alternatively, the network can be a single personal computer, a hospital virtual private network, or a local intranet. Further, the network and software can be integrated with a hospital PACS system or partially loaded onto the imaging equipment itself such as an ultrasound device. The software can communicate over the network via email if necessary or accessed without any specific software only requiring a physician or other user to have an Internet browser capability.

The present invention is further directed to a method for credentialing a physician to use a medical imaging device including the following steps: 1) a physician conducts patient examination with the imaging device; 2) the physician views & stores digital medical images with the imaging device; 3) the imaging device transfers the digital images to a storage device; 4) the software loads the images; 5) the software encrypts the stored images; 6) the physician deletes images not required for training; 7) the physician splits-up image folders for training; 8) the physician selects an exam template by exam type; 9) the physician enters findings; 10) the physician submits an exam for review; 11) the software then de-identifies patient data; 12) a reviewer then selects physician exams to review; 13) the software de-encrypts submitted images; 14) the reviewer indicates findings as correct or incorrect; 15) the reviewer provides technical feedback on device usage and the related medical interpretations including annotations to the image itself; 16) the reviewer accepts or rejects each exam submitted; 17) the software then returns the exam and feedback to the physician; 18) the physician checks on the system for feedback; 19) the physician reviews and acknowledges feedback electronically; 20) the physician completes 25-50 exams for each standard exam type; 21) the physician completes non-standard exams (if any); and 22) the physician completes quality assurance exams required (if any). In an exemplary embodiment, these steps may be performed, e.g., in approximately the same sequence presented above. Additional steps include an administrator 23) preparing templates for 6 standard exam types; 24) preparing templates for non-standard exam types; 25) specifying the range and frequency of exams required; 26) indicating authorized physicians and other users; 27) evaluating progress and evidence of completion; and 28) compiling statistics and research data to enhance and expand the review and quality assurance templates for the future. Further, the system can create credentialing letters and study documentation and lists to evidence completion of credentialing requirements.

Alternatively, the method may be modified if the system is partially integrated into the imaging device itself and includes the following steps: 1) a physician conducts a patient examination with the imaging device; 2) the physician views & stores digital medical images with the imaging device; 3) the physician selects an exam template by exam type with the imaging device; 4) the physician deletes images not required for training with the imaging device; 5) the physician splits-up image folders for training with the imaging device; 6) the physician enters findings with the imaging device; 7) the physician submits the exam for review directly from the imaging device; 8) the imaging device then encrypts the stored images (for review); 9) the imaging device de-identifies patient data; 10) a reviewer then selects physician exams to review; 11) software de-encrypts images submitted; 12) the reviewer indicates findings as correct or incorrect; 13) the reviewer provides technical feedback on device usage including annotations to the image itself; 14) the reviewer accepts or rejects each exam submitted; 15) the system then returns the exam and feedback to physician; 16) the physician then checks on the system for feedback; 17) the physician reviews and acknowledges feedback electronically; 18) the physician completes 25-50 exams for each standard exam type; 19) the physician then completes non-standard exams (if any); and 20) the physician also completes quality assurance exams required. In an exemplary embodiment, these steps may be performed, e.g., in approximately the same sequence presented above. Additional steps include an administrator 21) preparing templates for 6 standard exam types; 22) preparing templates for non-standard exam types; 23) specifying the range and frequency of exams required; 24) indicating authorized physicians and other users; 25) evaluating evidence of completion; and 26) compiling statistics and research data to enhance and expand the review and quality assurance templates in the future. Further, the system can create credentialing letters and study documentation and lists to evidence completion of credentialing requirements.

According to an exemplary embodiment of the present invention, a method for credentialing a test taker to use a device includes: outputting a first representation of an electronic exam according to a first view if the exam is accessed by a test taker, the electronic exam including data associated with a use of a device; and outputting a second representation of the exam according to a second view if the exam is accessed by a test reviewer.

The method may further include storing the exam in a memory.

The device may be a diagnostic device including, for example, an X-ray device, a magnetic resonance imaging device, an ultrasound device, a computer tomography device, a computer radiography device, a mammography device, or a nuclear medicine device.

The method may further include appending diagnostic data output by the device during the use to the exam.

The diagnostic data may include, for example, one or more images.

The method may further include: outputting a plurality of images; dividing the plurality of images into a plurality of image sets, each image set corresponding to at most one corresponding exam; and deleting an image of the plurality of images that is determined to be unnecessary for any exam. The dividing and the deleting may be performed in response to corresponding user instructions.

The image set may be appended to the exam.

The data associated with the use of the device may include a diagnosis rendered based on the appended image set and/or a measurement associated with the appended image set.

The method may further include encrypting the image set, the diagnosis, and/or the measurement. The encrypting may be performed by using a single encryption key and/or HTTPS including double key encryption for Internet transmissions to ensure maximum security for any sensitive patient information.

The plurality of images may be output at the device, and the method may further include: downloading the plurality of images from the device onto a storage device; and uploading the plurality of images from the storage device to a system in which the electronic exam is stored.

The method may further include storing data format information for a device manufacturer.

The uploading may include matching a format of the plurality of images to a stored data format of the data format information, and may be performed in accordance with the matched format.

Outputting the representations may include displaying the representations on a graphical user interface. The data associated with the use of the device may include first data appendable to the exam when the first representation is displayed and second data appendable to the exam when the second representation is displayed. The second data may include an indication of whether the exam is a passing exam.

The method may further include generating the exam by instantiating a template that includes a particular data designated for output at least in the first representation. The particular data may be a request for input from a physician.

The data associated with the use of the device may include an identification of a patient who is a subject of the use. The identification may be viewable in the first representation and not viewable in the second representation. The identification may be embedded in an output of the diagnostic device that is appended to the exam. The method may further include: generating a copy of the output of the diagnostic device; removing the identification from the output copy; and subsequently storing the output copy. The output of the diagnostic device may be viewable in the first representation and the output copy may be viewable in the second representation.

The data associated with the use of the device may include a first data that includes the identification and that is viewable in the first representation and not viewable in the second representation. The method may further include: generating a copy of the first data; removing the identification from the copy; and storing the copy as a second data viewable in the second representation.

The method may further include: providing a plurality of selectable stored exams, including the electronic exam discussed above. The second representation may be output when the exam is selected by the reviewer. The plurality of selectable stored exams may include exams indicated to have been taken by a plurality of test takers. Each of the plurality of selectable stored exams may be selectable by the reviewer. An exam of the plurality of selectable stored exams may be selectable by the test taker upon a condition that it is indicated to have been taken by the test taker.

The method may further include generating a report of statistics. The report may be based on exam results of the plurality of selectable stored exams.

The plurality of exams may be sorted or sortable by exam type. The method may further include displaying a plurality of exam folders. Each folder may correspond to one of a plurality of exam types and may include a corresponding subset of the plurality of exams. The corresponding subset may include exams of only an exam type of the corresponding folder. [00971 The plurality of exam types may include standard exam types and/or custom exam types. The plurality of exam types may include, for example, an OB-GYN exam type, a gall bladder exam type, an aorta exam type, a cardiac exam type, a trauma exam type, a kidney exam type, a lung exam type, a central venus access exam type, and/or a DVT exam type.

A subset of the plurality of selectable stored exams may be associated with the test taker. The method may further include dividing the subset into a plurality of selectable exam groups. The groups may include, for example, a completed exams group, a submitted exams group, a pending exams group, a feedback ready group, a passed exams group, and/or a failed exams group.

The electronic exam may be an interactive form. The data associated with the use of the device may include a first data enterable in the first representation and not in the second representation and a second data enterable in the second representation and not in the first representation. The first and second data may be viewable in the first and second representations. The second data may include an indication of whether the exam is a passing or a failing exam. The method may further include: generating for the test taker test taking results that indicate a number of passed exams and/or a number of failed exams; and updating the results based on the indication. The method may further include: storing for each of at least one entity a corresponding file indicating credentialing requirements of the entity; for the test taker, determining, for each of one or more of the at least one entity and based on indicated credentialing requirements and the results, whether the test taker is credentialed, a number of credentialing requirements satisfied, at least one type of credentialing requirement satisfied, a number of credentialing requirements unsatisfied, and/or at least one type of credentialing requirement unsatisfied; and including one or more of the determinations in the results.

The credentialing requirements may include a required number of passed exams for each of one or more exam types and/or a frequency of test taking or test passing for each of the one or more exam types.

The method may further include generating a report based on the results.

The method may further include storing a plurality of exam templates. The method may further include generating the electronic exam by instantiating an exam template. One or more of the templates may be modifiable. The method may further include displaying a template edit form for generating and/or editing a particular exam template. The form may include a plurality of manipulatable fields for customizing the particular exam template. The fields may include at (a) an exam type identification field, (b) a user type identification field indicating to which of a plurality of representations of a particular exam the particular exam template corresponds, each representation associated with a corresponding user type, (c) a category field, and/or (d) one or more subcategory fields. The fields may include user definable fields. The fields may include at least one field corresponding to a field of the electronic exam.

According to an example embodiment of the present invention, a method for credentialing a user to use a device may include: in response to a first input that is input by a first user identified as a test taker, storing an electronic exam including data associated with a use of a device; and in response to a second input that is input by a second user identified as a test reviewer, outputting at least a portion of the electronic exam.

The device may, for example, be a diagnostic device.

The method may further include modifying the electronic exam to include data input by the second user. In an embodiment, at least a portion of the data input by the second user may be viewable by the first user when the electronic exam is subsequently output to the first user.

The electronic exam may be stored in a memory unit of the diagnostic device.

The electronic exam or different portions of the electronic exam may be viewable by the first and second users at a same output device.

The electronic exam may be transferred between the first and second users over a network. The method may further include: encrypting an entire or at least a portion of the electronic exam prior to a transmission of the electronic exam over the network; and decrypting the entire or a portion of the electronic exam when it is opened at a receiving end of the transmission using a correct password and encryption key or keys.

The electronic exam may include at least one interactive form modifiable by the first and second users.

According to an example embodiment of the present invention, an electronic exam for diagnostic device use credentialing may include an instantiated data structure. The data structure may include at least one field for an appended output of a diagnostic device, at least one field for input of a first data that is determined based on the output and that is indicated to be input by a first user, and at least one field for evaluation data of the first data that is indicated to be input by a second user.

According to an example embodiment of the present invention, a system for diagnostic device use credentialing may include: an arrangement for instantiating an exam template to generate an exam; an arrangement for appending output of a diagnostic device to the exam; an arrangement for modifying data of the exam in response to input by a first user logged into the system as a test taker; an arrangement for receiving an instruction from a second user logged into the system as a reviewer to output the exam; an arrangement for outputting the exam and/or a portion of the exam in response to the instruction; and an arrangement for modifying data of the exam in response to input by the second user. At least a portion of data input by the first user may be viewable by the second user and at least a portion of data input by the second user may be viewable by the first user.

According to an example embodiment of the present invention, a computer-readable medium may have stored thereon instructions which, when executed, perform a diagnostic device use credentialing method. The method may include: in response to a first input that is input by a first user identified as a test taker, storing an electronic exam including data associated with a use of a diagnostic device; and in response to a second input that is input by a second user identified as a test reviewer, outputting at least a portion of the electronic exam.

According to an example embodiment of the present invention, a diagnostic device may include: an arrangement for outputting diagnostic data; and an arrangement for appending the diagnostic data to an electronic diagnostic device use credentialing exam.

The diagnostic device may be an imaging device and the diagnostic data may represent at least one image.

The diagnostic device may further include an arrangement for instantiating a template to generate the exam.

The diagnostic device may further include an arrangement for modifying data of the exam in response to input by a first user logged into a system of the diagnostic device as a test taker.

The diagnostic device may further include an arrangement for providing or transmitting the exam including its appended data to a second user logged into the system as a reviewer.

The exam or a portion of the exam may be output in response to an instruction input by the second user. In one embodiment, the diagnostic device may further include an arrangement for receiving the instruction from the second user and outputting the exam or portion of the exam in response thereto.

The diagnostic device may include an arrangement for outputting to the first user data included in the exam by the second user.

According to an example embodiment of the present invention, a method for credentialing an exam taker to use a device, for example, a diagnostic device, may include: a) posing test questions to the exam taker; b) sending to a reviewer (i) the exam taker's answers to the test questions and (ii) data associated with the device to which the test questions relate; c) posing review questions to the reviewer relating to the exam taker's answers; and d) sending answers of the reviewer to the review questions to the test taker, where at least one of the posing steps (a) and (c) is done electronically.

In one embodiment, at least one of the sending steps (b) and (d) may be done over a network. Further, the data associated with the device to which the test questions relate may be presented differently to the exam taker and the reviewer.

According to an example embodiment of the present invention, a system for credentialing an exam taker to use a device, for example, a diagnostic device, may include arrangements for a) posing test questions to the exam taker in an electronic form; b) sending to a reviewer (i) the exam taker's answers to the test questions and (ii) data associated with the device to which the test questions relate; c) posing review questions in electronic form to the reviewer relating to the exam taker's answers; and d) sending answers of the reviewer to the review questions to the test taker.

The system may include an input device, an output device, an imaging device, a processor, and a memory device.

In one embodiment, at least one of the sending steps (b) and (d) may be done over a network. The data associated with the device to which the test questions relate may be presented differently to the exam taker and the reviewer.

These and other aspects of the present invention will become apparent to those skilled in the art after a reading of the following description of the preferred embodiment when considered with the drawings.

### BRIEF DESCRIPTION OF THE DRAWINGS

FIG. 1 is a flow chart illustrating a conventional procedure performed with respect to an existing system.

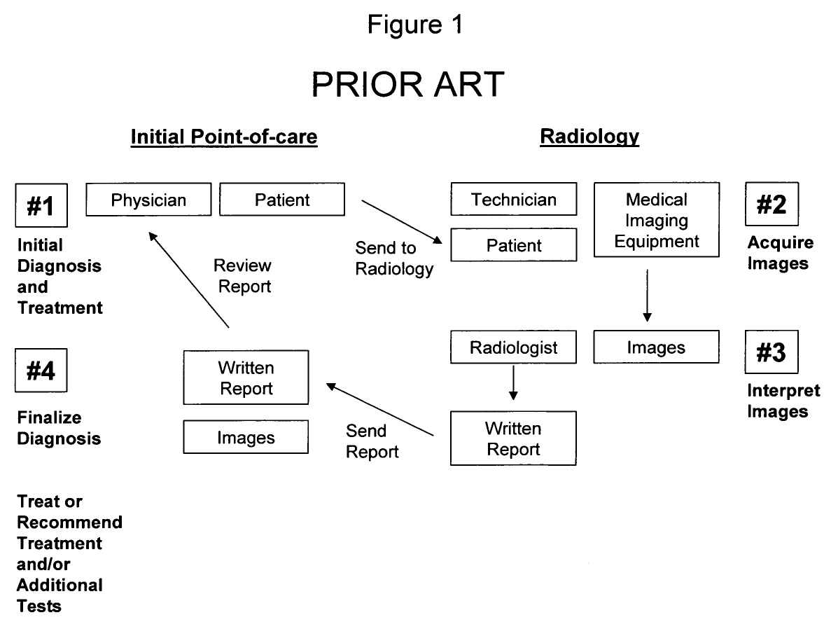

FIG. 2 is a diagram of a conventional ultrasound device.

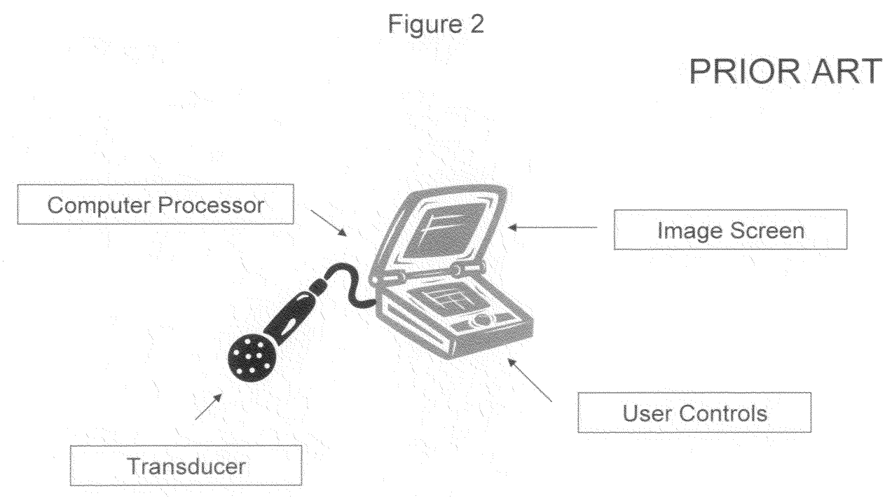

FIG. 3 is a diagram illustrating conventional use by a physician of a mobile medical imaging device to diagnose and/or treat a patient at the initial point of care.

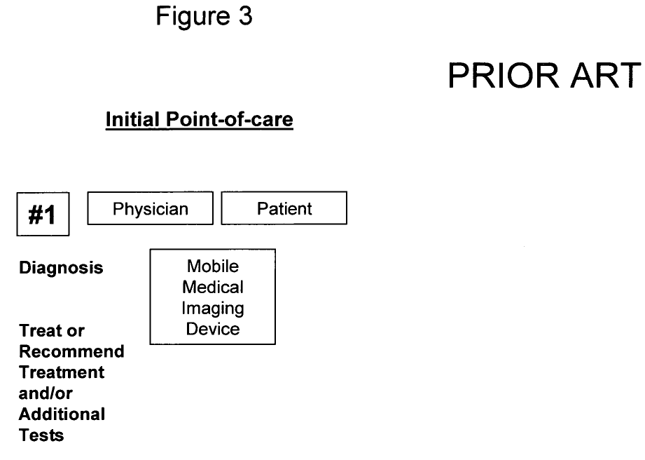

FIG. 4 is a diagram of a computer-based review and quality assurance system being utilized by individual physicians according to the present invention.

FIG. 5 is a diagram of a computer-based review and quality assurance system being utilized by multiple users in a hospital or health institution according of the present invention.

FIG. 6 is a diagram of a computer-based review and quality assurance system being utilized by a physician's office or clinic according of the present invention.

FIG. 7 is a diagram of the major components of the computerized software according present invention.

FIG. 8 is a flow chart of the method according the present invention.

FIG. 9 is a screen view of a graphical user interface for a physician according to the present invention.

FIG. 10 is a screen view of a graphical user interface for a super administrator according to the present invention.

FIG. 11 is a diagram of the universal encryption provided for an individual physician according to the present invention.

FIG. 12 is a diagram of the universal encryption provided for multiple users in a hospital or health institution according to the present invention.

FIG. 13 is a screen view of a graphical user interface presenting images for a physician according to the present invention.

FIG. 14 is a screen view of a graphical user interface for a physician presenting image manipulation according to the present invention.

FIG. 15 is a sample of a standardized exam template according to the present invention.

FIG. 16 is a screen view of the physician's graphical user interface for the exam template according to the present invention.

FIG. 17 is a screen view of the reviewer's graphical user interface for the exam template according to the present invention.

FIG. 18 is a diagram of template examples according to the present invention.

FIG. 19 is a screen view of exam templates according to the present invention.

FIG. 20 is a screen view of the super administrator's graphical user interface for managing an exam template for physician questions according to the present invention.

FIG. 21 is a screen view of the super administrator's graphical user interface for managing an exam template for reviewer feedback according to the present invention.

FIG. 22 is a screen view of the super administrator's graphical user interface showing categories of answers and feedback according to the present invention.

FIG. 23 is a screen view of the reviewer's graphical user interface showing allowable answers and feedback according to the present invention.

FIG. 24 is a screen view of the administrator's graphical user interface showing the required numbers of exams according to the present invention.

FIG. 25 is a screen view of the physician's graphical user interface for tracking progress towards credentialing requirements according to the present invention.

FIG. 26 is a diagram of an alternative embodiment of the computer-based review and quality assurance system being utilized by a single hospital department according to the present invention.

FIG. 27 is a diagram of an alternative embodiment of the computer-based quality assurance system being utilized by a hospital and accessed over a virtual private network according to the present invention.

FIG. 28 is a diagram of an alternative embodiment of the computer-based quality assurance system integrated with a PACS being utilized by a hospital and accessed over a virtual private network according to the present invention.

FIG. 29 is a diagram of an alternative embodiment of a computer-based quality assurance system that is partially integrated within a medical imaging device according to the present invention.

FIG. 30 is a flow chart for an alternative embodiment of the method according to the present invention.

FIG. 31 is a diagram of an alternative embodiment of a computer-based review and quality assurance system being utilized by individual physicians and communicating with a reviewer over email according to the present invention.

FIG. 32 is another screen view of the physician's graphical user interface for the exam template according to the present invention.

FIG. 33 is another screen view of the physician's graphical user interface for the exam template according to the present invention.

FIG. 34 is a diagram showing two layer data encryption of at least one dataset according to the present invention.

FIG. 35 is a diagram laying out a dataset according to the present invention.

FIG. 36 is an example of a medical exam template form for data capture for an aorta focused examination according to the present invention.

FIG. 37 is an example of a medical exam template form for data capture for a cardiac focused examination according to the present invention.

FIG. 38 is an example of a medical exam template form for data capture for a biliary focused examination according to the present invention.

FIG. 39 is an example of a medical exam template form for data capture for a trauma focused examination according to the present invention.

FIG. 40 is an example of a medical exam template form for data capture for an OB/GYN focused examination according to the present invention.

FIG. 41 is an example of a medical exam template form for data capture for an other/trauma focused examination according to the present invention.

FIG. 42 is an example of a medical exam template form for feedback data for an aorta focused examination according to the present invention.

FIG. 43 is an example of a medical exam template form for feedback data for a cardiac focused examination according to the present invention.

FIG. 44 is an example of a medical exam template form for feedback data for a biliary focused examination according to the present invention.

FIG. 45 is an example of a medical exam template form for feedback data for a trauma focused examination according to the present invention.

FIG. 46 is an example of a medical exam template form for feedback data for an OB/GYN focused examination according to the present invention.

FIG. 47 is an example of a medical exam template form for feedback data for an other/trauma focused examination according to the present invention.

### DETAILED DESCRIPTION OF THE PREFERRED EMBODIMENTS

In the following description, like reference characters designate like or corresponding parts throughout the several views. Also in the following description, it is to be understood that such terms as “forward,” “rearward,” “front,” “back,” “right,” “left,” “upwardly,” “downwardly,” and the like are words of convenience and are not to be construed as limiting terms.

### Overview

Referring now to the drawings in general; the illustrations are for the purpose of describing a preferred embodiment of the invention and are not intended to limit the invention thereto. The present invention is a computer-based system and method that permits training and supplying of feedback to physicians who wish to become proficient in the use of handheld ultrasound and use of other medical imaging devices to directly, efficiently, effectively, and quickly diagnose patients at the initial point-of-care.

The system and method provides a single integrated facility for physicians to collect and prepare medical images they acquire during medical examinations of actual patients performed for training purposes and to submit these exams to qualified reviewers who provide feedback needed for the physicians to become credentialed to use the imaging equipment for future non-training examinations thereafter. The system and method combines feedback simultaneously for two distinctively different types of activities discussed above, namely the A) use of medical imaging equipment and B) interpretation and evaluation of images to make a diagnosis.

Generally, the system according to the present invention includes imaging equipment, software, and a network. The imaging equipment may have multiple modalities, is used at the point of care, incorporates digital image files, and is used for credentialing and quality assurance. The system is designed to work with, for example, ultrasound, MRI (magnetic resonance imaging), CT scan, and X-ray imaging technology. The system may be optimized for mobile or portable imaging equipment, such as ultrasound scanners. At the point of care, portable imaging equipment creates a new, image-based form of a patient medical examination, which allows for a physician to directly use and analyze the images instead of referring the patient to radiology. Whereas medical images used to be film-based, new images are digital, which can be sent electronically. Therefore the present invention provides a system to take advantage of these developments to credential physicians more efficiently in the use of portable imaging at the point of care.

Physicians should be credentialed to use imaging equipment. Generally, the process involves the physician capturing the images with the portable imaging device in a live clinical setting, sending both the images and diagnostic findings to a qualified reviewer, and then receiving feedback from the reviewer. The feedback is centered on the use of the equipment and the interpretation of the images.

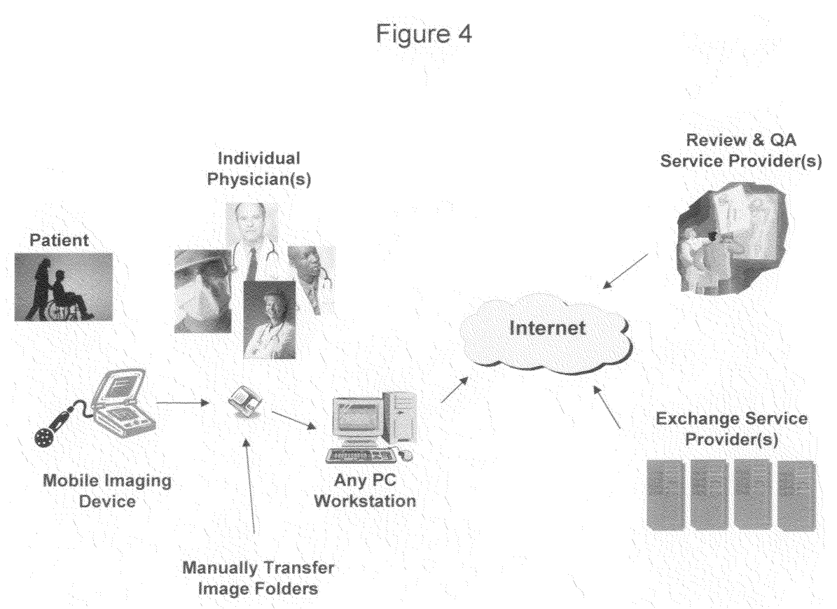

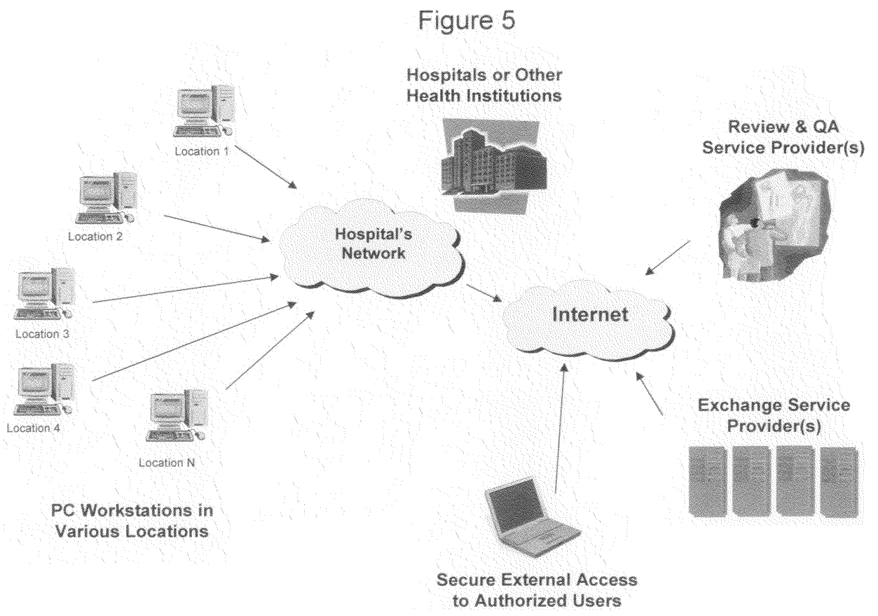

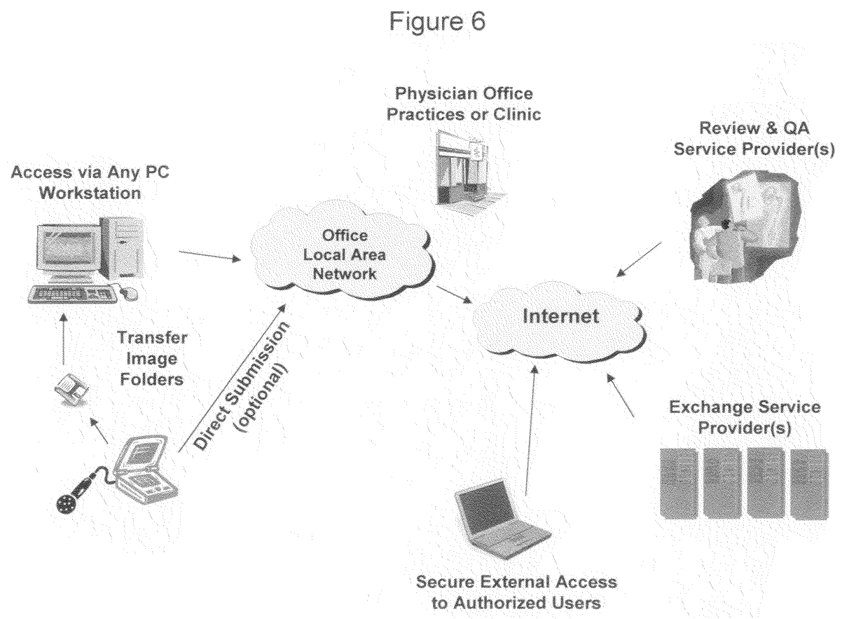

An overview of the system of the present invention is illustrated in FIGS. 4, 5, and 6. Preferably, as shown in FIG. 4, individual physicians can access an Internet service using the system to securely submit exams including both images and diagnostic findings for feedback and review. Alternatively, as shown in FIG. 5, physicians can use the system in different locations throughout a larger hospital or other health institution. Alternatively, as shown in FIG. 6, physicians can use the system in a single office practice or clinic.

With the exception of medical teaching institutions, the current options include use of either paper-based forms or videotapes including typically sending these materials out via the mail. Even at selected teaching institutions where there is strong expertise in the use of mobile image technology, the approach still involves use of paper-based forms with the only advantage being that reviewers are available on the premises.

To solve many of the problems with the prior art, the present invention is directed toward a system and method for credentialing physicians to use mobile imaging devices at the initial point of care. This credentialing process generally requires a physician to assemble and receive feedback on anywhere from 150-300 ultrasound exams including exams performed across all standard exam types needed for this credentialing process (e.g., aorta, cardiac, gall bladder, kidney, OB-GYN, trauma). The number of exams required for each exam type and cumulatively across types depends on how a hospital or other health institution chooses to apply well established guidelines, such as those from ACEP.

### Software

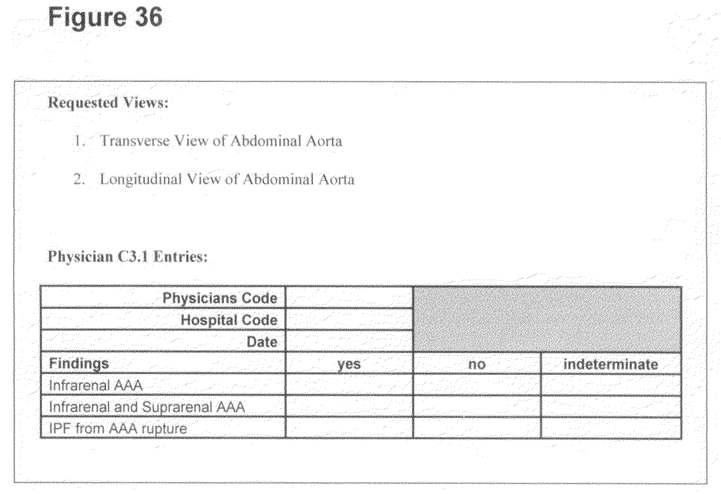

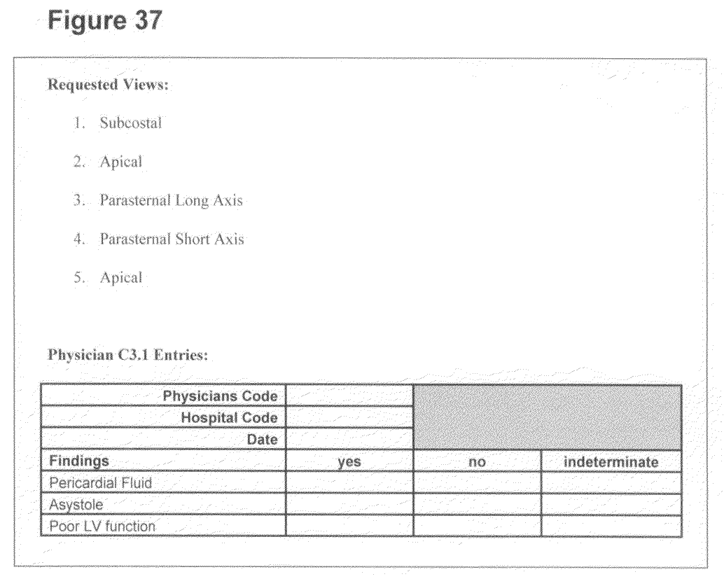

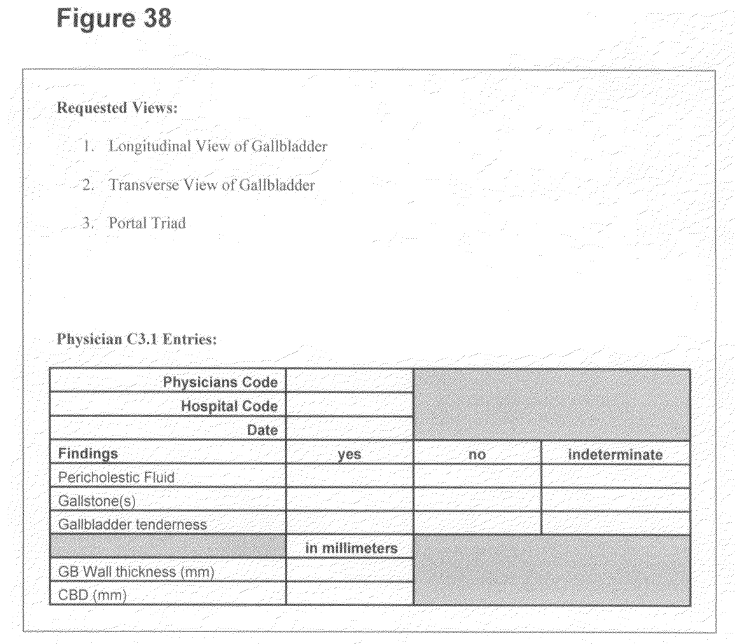

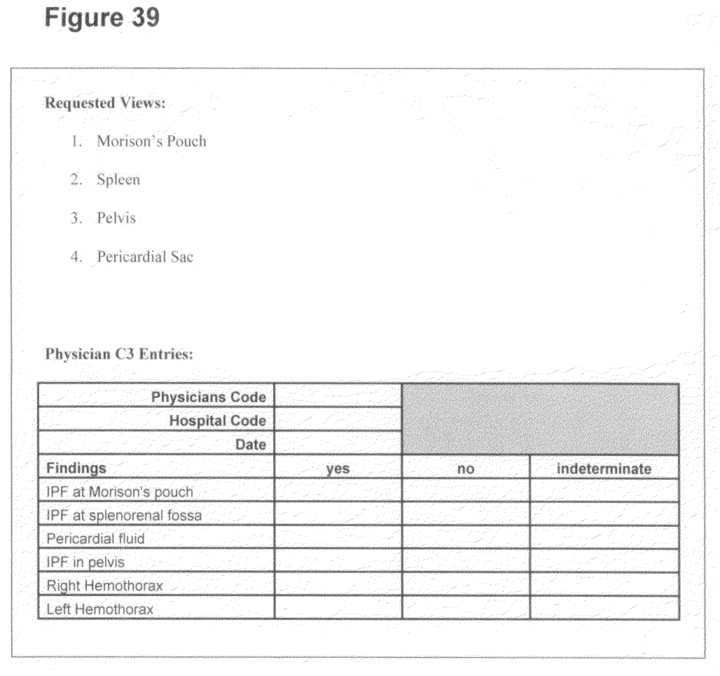

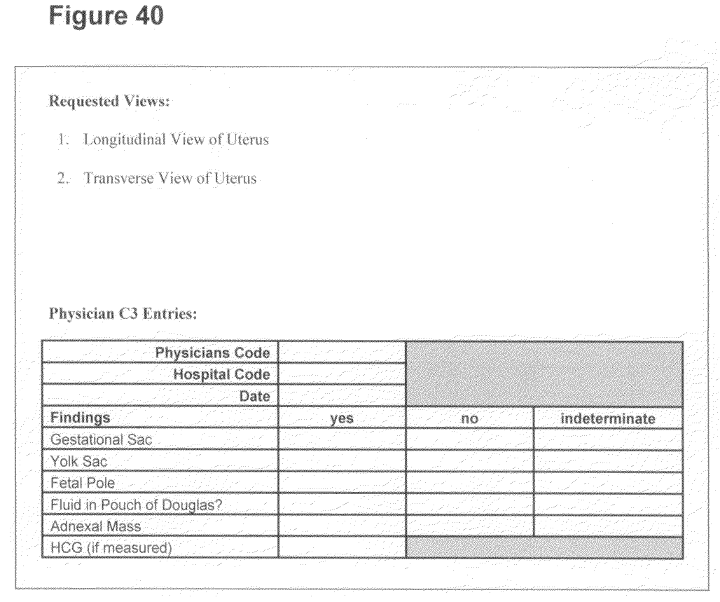

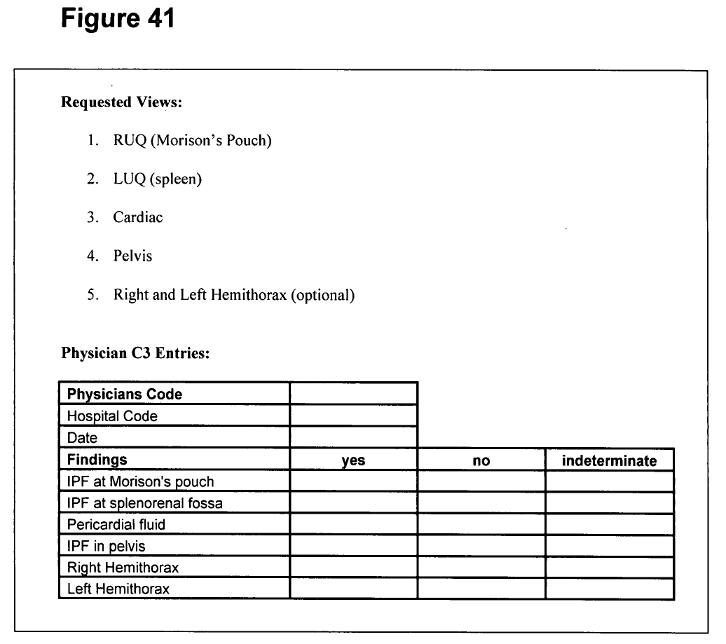

To facilitate the credentialing process, the present invention uses software that generally includes a standardized exam template, image folder load tables, universal encryption, various user views, message management, and a universal directory. A workstation review system within the software supports both local and remote reviewers. A physician submits, in a template form, the images and diagnostic findings based on an exam type. The template form is very specific for each exam type, including a limited number of questions regarding the key medical findings and specific image views required, correct images views acquired, quality of images, and if the image-based exam is acceptable or unacceptable to a reviewer (see, for example, FIGS. 36-41).

There are seven common medical examinations including, aorta, cardiac, gall bladder, kidney, lung, trauma, and obstetrics-gynecology although more exam types will likely evolve in the future. The image folder load tables allow for editing of duplicates and automatic loading. The media can be transferred via floppy disk, USB memory stick or other memory transfer device, or through direct automated transfers. Universal encryption utilizes two different levels of encryption both of which can be found in many software programs and/or is commonly used for transferring sensitive data over the Internet.

Within the software there are alternative views for a physician, reviewer, local administrator, and super administrator. The physician is able to split folders based on exam type, delete extra images, and manipulate images. The reviewer marks the physician's medical findings as correct or incorrect, annotates the images, and assigns an overall acceptability rating for each image-based exam. Message management allows for sent exams to be acknowledged, for missing exams to be sent, and to avoid storing duplicate exams. A Universal Directory issues user IDs and passwords, and issues serial numbers, encryption keys and digital certificates for users who wish to send exams over the Internet for review using an Exchange Service Provider.

Duplicate exams are detected when a physician attempts to load a folder with a name that is identical to a folder previously loaded by that same physician. Generally, there should be from 150 to 400 folders required for credentialing including a reserve for extra folders needed if exams are not deemed acceptable by a reviewer for credentialing purposes. Preferably, the physician has an option to override a warning from the system that he or she is attempting to load a duplicate folder name if there is a legitimate reason. As such, the preferred embodiment provides for some level of control against a physician inadvertently submitting the same exams repeatedly to count towards credentialing.

Automatic loading involves maintaining a table of the different file structures maintained by different ultrasound vendors for manually downloading medical image folders. When a physician attempts to load image folders, the system tests all of the different known vendor formats until it recognizes the format for the particular vendor ultrasound or other imaging, device from which the images were downloaded. The system then proceeds to load all the folders using the format to download image folders used by the vendor (and uses this same format to display the folders).

There is a standard for transferring and managing medical images and related data called DICOM. However, DICOM is not universally applied for manually downloaded medical images. Even in cases where DICOM is applied there are often subtle differences in how vendors interpret the DICOM standards and how users invoke different options for downloading folders under these standards creating differences in the resulting folder data formats. As such, the present invention affirmatively handles any of these situations in reading medical image folders loaded by the physician or through other means (e.g., automated interfaces).

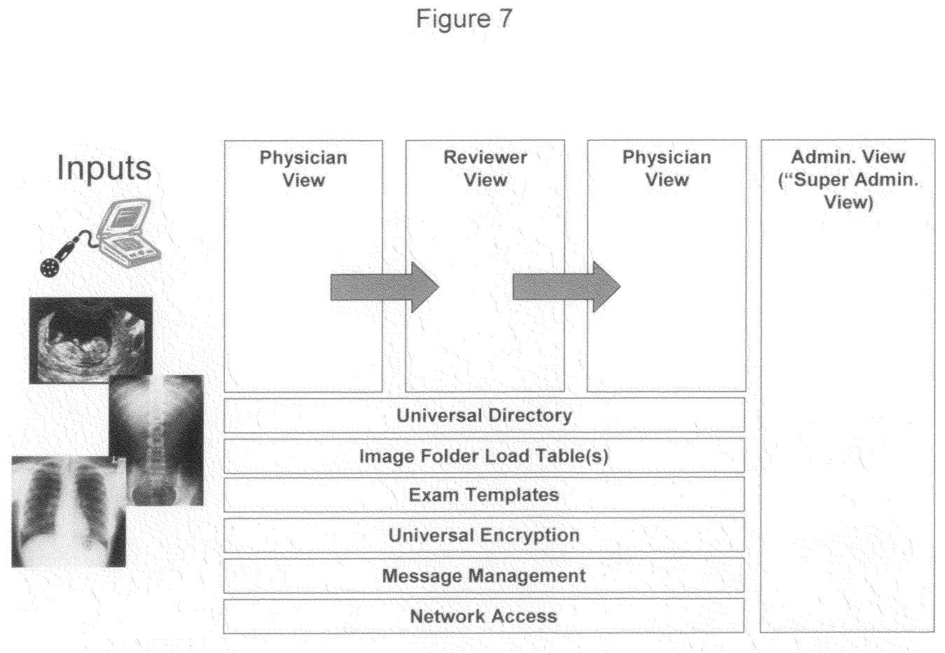

FIG. 7 shows the major components of the software according to the present invention. The software preferably includes different “views,” along with several underlying functions used to integrate the different views into a single integrated system and method for performing image exam reviews and quality assurance (QA). These functions include a Universal Directory, Image Folder Load Tables, Exam Templates, Universal Encryption, Message Management and Network Access. Each of the views and underlying functions are described throughout this section.

For the preferred embodiment, the software would be enabled by an Exchange Service Provider as shown in FIGS. 4, 5 and 6. The software would be issued with the following:
A. Encryption key
B. Unique serial number for software issued
C. Individual User or Group ID and password

The software is preferably loaded onto one or more standard Windows-based workstation to be used for the review and QA process. This type of software is commonly referred to as a “fat” or “rich” client (versus remote web-based computer applications that can be accessed using a PC workstation using only a “thin” client web browser, e.g. Microsoft Internet Explorer, without having to load any type of additional “fat” or “rich” client software). Alternatively, the software can be implemented to run on other operating systems such as Linux, MacOS, UNIX, etc. Finally, the software could be configured so that a physician or other user could access it with only an Internet browser or so called “thin” client web browser.

An Exchange Service Provider facilitates the secure exchange of exams between physician users or groups (hospitals) and reviewers. This is a process that traditionally would be difficult or impossible for these users and reviewers to facilitate on their own without some type of intermediary. As such, the Exchange Service both distributes software if needed and facilitates the secure exchange of exams over the Internet for the purpose of conducting credentialing reviews. In cases where a hospital or other health institution does not need to go outside to obtain review services, use of the Exchange Service Provider would not be necessary (i.e., for institutions who are capable of credentialing their physicians internally without the support of an outside reviewers or a review service).

Users can only operate the software if they use a correct User ID and password. User ID's and passwords are preferably issued by the Exchange Service Provider for the system as pictured in FIG. 4 (i.e., for individual physician users of the service over the Internet). Alternatively, user ID's and passwords may be issued by the Hospital, Physician Office or Clinic for their own internal users as pictured in FIGS. 5 and 6.

A user who is authorized to access the software is also able to open the image files and folders stored using the software with the same serial number. However, any user who tries to access someone else's images using the same review and QA software that has a different serial number and encryption key will not be able to open the other user's image folders. Additionally, physicians are restricted from opening any exam folders except their own. Authorized administrators and reviewers though can view exam folders generated by the same release of the software or that are conveyed to them by an Exchange Service Provider for authorized purposes.

Preferably, the software is issued with a user ID, password, and a unique software release serial number or an encryption key or both. As such, only the user or users authorized to use a specific release of the software are able to open image folders and exams created with that software. So for instance, if two different hospitals buy the same software and transfer exam files between each other they will not be able to open those files; thus patient privacy can be maintained within the institution that is authorized to handle that patient.

Additionally, if two physicians sign-up for a credentialing review service over the Internet and transfer exam folders between each other, they will not be able to look at the patient folders received from the other physician without going through the Exchange Service to appropriately authorize access. As such, the only exception is when a “trusted source” (i.e., the Exchange Service Provider) that originally issued the encryption key and software is used to open an exam folder it receives from a user to whom the trusted source originally issued the software for the purpose of exchanging this folder with an authorized reviewer.

So for instance, the trusted source will be able to receive exam files from physicians, decrypt them so that an authorized reviewer can review them. As such, there are 2 and potentially 3 pieces of information needed to open any exam file—a unique user ID and password of the individual user or group to whom the software was issued, and the unique software serial number or the encryption key or both. Additionally, a second layer of security will be put in place. The software will only allow transmission of the image and date over the Internet using an additional secure HTTPS protocol involving double key encryption implemented using digital certificates issued by a trusted source (in the case of the preferred embodiment, this is issued by the Credentialing Service Provider).

An Exchange Service Provider, as a trusted source, can open any image folder supplied to it by any user of the software regardless of the serial number. As such, the Exchange Service is able to open any folder and provide it to authorized reviewers as needed to facilitate remote image reviews and QA. There are numerous security and privacy concerns in healthcare and the above approach guards against unauthorized individuals from accessing medical information (even if that information is used only for training purposes). Additionally, The Exchange Service Provider holds all of the Encryption Keys and Digital Certificates for each user and the unique serial number assigned to each customer's release of the software.

The preferred embodiment contains one final security precaution. When physicians view their own exam folders the image and the folder name may in fact contain the patient name and patient ID. However, when the exam folders are conveyed to the reviewer, this patient information appearing in the exam folder will be removed or “de-identified”. This is a common term used in the healthcare industry. For the preferred embodiment, it refers to a process of removing any specific reference to the patient name or any other information that could be used to identify the patient. There are two aspects of this process. First, patient names and ID's often appear on the images themselves. But this information typically appears in the same standard location in the images acquired for each particular image equipment vendor or vendor equipment model number. As such, the preferred embodiment will blank out this information based upon the standard location where this information appears for each different vendor or vendor model image format. Second, patient information may be reflected in the naming of the image exam folder. In this regard, the preferred embodiment will present the actual folder name to the physician that contains the patients name but will present a machine generated folder name for reviewers.

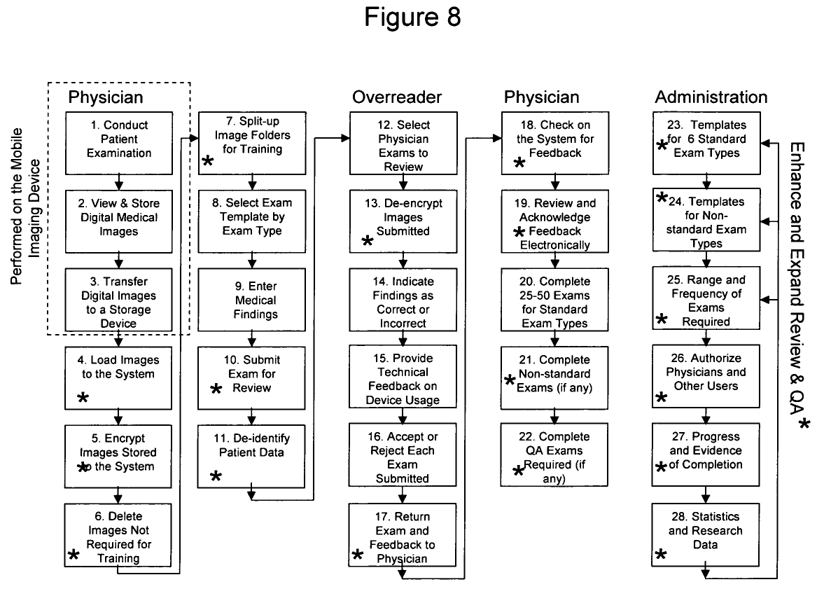

Further, the software preferably includes a series of administrative functions necessary to support the review process, as shown in FIG. 8 (boxes 23-28). Preferably, these include processes for creating and maintaining image exam “templates” for both the 6 standard exam types (box 23 in FIG. 8) and other non-standard exam templates (box 24) that can be customized for example for local hospital needs or new future exam types developed. Additionally, in box 25 the range of exams required is entered based on current credentialing standards (e.g., anywhere 25-50 aorta exams, 25-75 OB-GYN exams, etc.); also entered at this step is the frequency required for post-credentialing quality assurance spot checking. These administrative processes can be either maintained by the Exchange Service Provider, by a group user such as a hospital or some sharing of these responsibilities across both parties.

The software also preferably enables an administrator to access the system to issue user ID's, passwords and encryption keys thereby authorizing physicians and other users to use the system (box 26.). The software enables tracking and reporting on progress in meeting credentialing and other quality assurance requirements (box 27) and collecting data on completed exams (e.g., questions most frequently missed, numbers of unacceptable exams, etc.) (box 27). This collected, empirical data can then be used to continuously refine and modify the exam templates, the numbers of exams required and the frequency of quality assurance procedures.

Moving back to FIG. 7, the Universal Directory function enables an Exchange Service Provider to issue user IDs and passwords for individual physician users or group users (such as hospitals) of the present embodiment who wish to exchange exam folder over the Internet for the purpose of conducting reviewer. The Universal Directory is also used to keep track of software serial numbers, encryption keys and digital certificates. The Universal Directory is a directory of all the individuals and groups to whom the software has been released. Each individual or group user is given a user ID, password, encryption key and unique software serial number. As a result, these users can use the services of an Exchange Service Provider to secure for instance a remote ultrasound review and QA services over the Internet.

For group users (e.g., a hospital), the group is given an encryption key and unique software serial number. However, the group is responsible for setting up its own individual users of their version of the system including setting up individual user ID's and passwords. Users on this unique version of the software are able to view exam files created by this version of the system but not exam files created by another entity or organization using the same software with a different serial number (individual physicians can only access their own exam folders, reviewers can access folders they are authorized to review, and a systems administrators can access all of the exam folders). These protections ensure patient data privacy and security within a given institution or by a specific physician are maintained by the software

As discussed above, a medical examination performed by a physician who is directly using a medical imaging device at the initial point of care is significantly different from traditional types of examinations involving a referring physician, image equipment technicians and radiologists or other imaging specialists. Therefore the review and QA process for training and credentialing physicians is also different from the traditional processes.

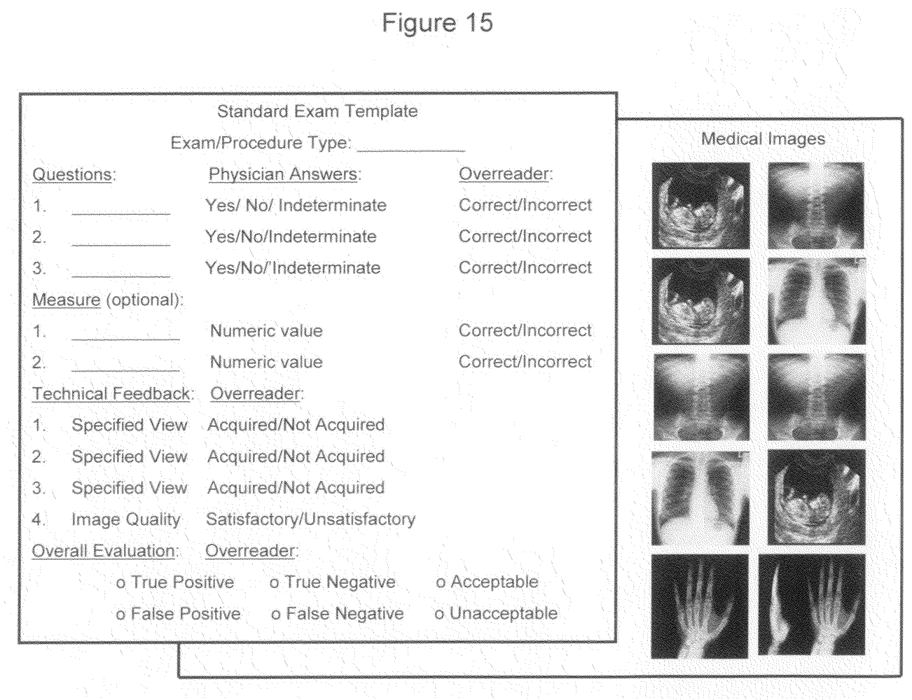

FIG. 15 shows the concept of a Standardized Exam Template for Physician Review and QA according to the present invention. The template preferably incorporates images and diagnostic findings from the physician and feedback from a reviewer that facilitates review of both of the following:
A. the use of medical imaging equipment; and
B. the accuracy of the physician's clinical findings based on the physician's acquired images.

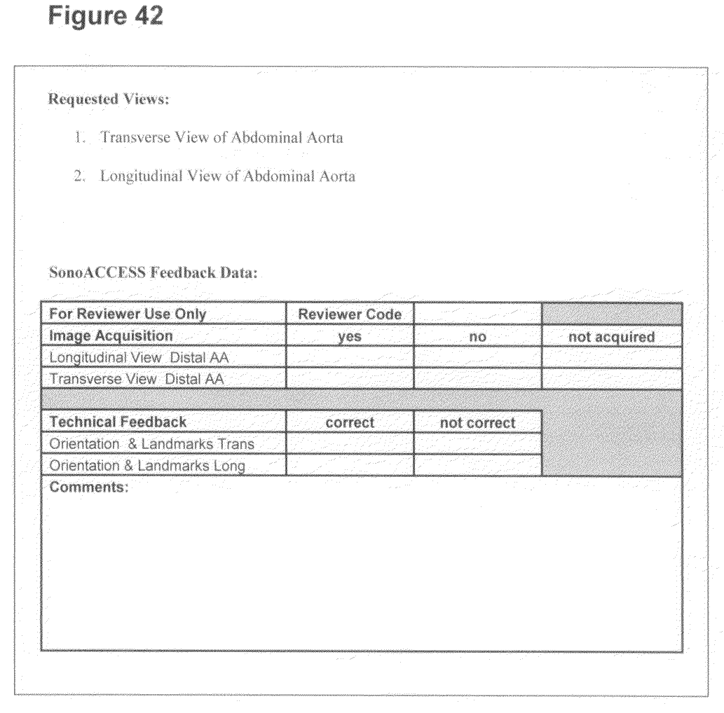

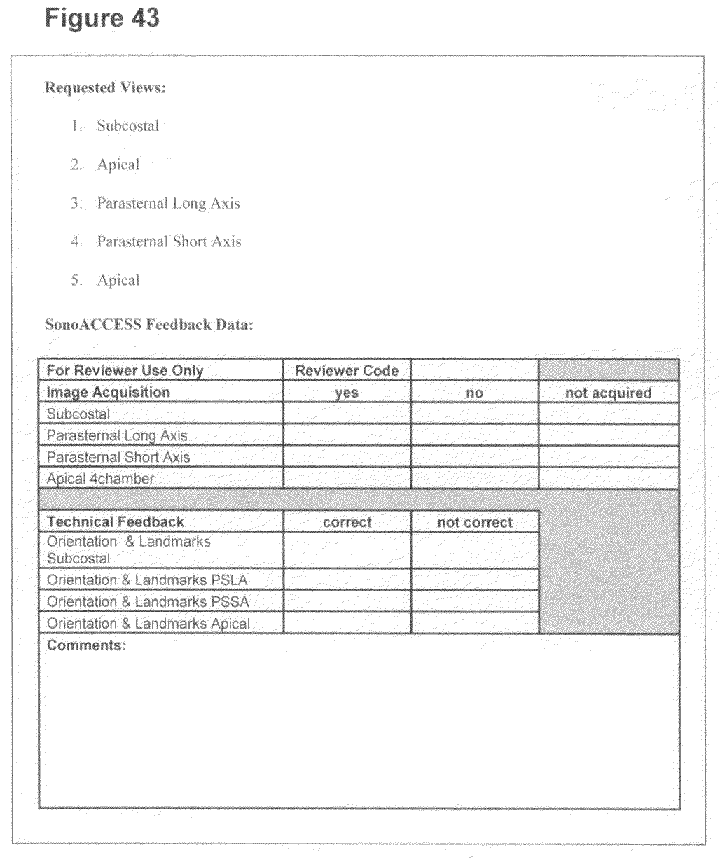

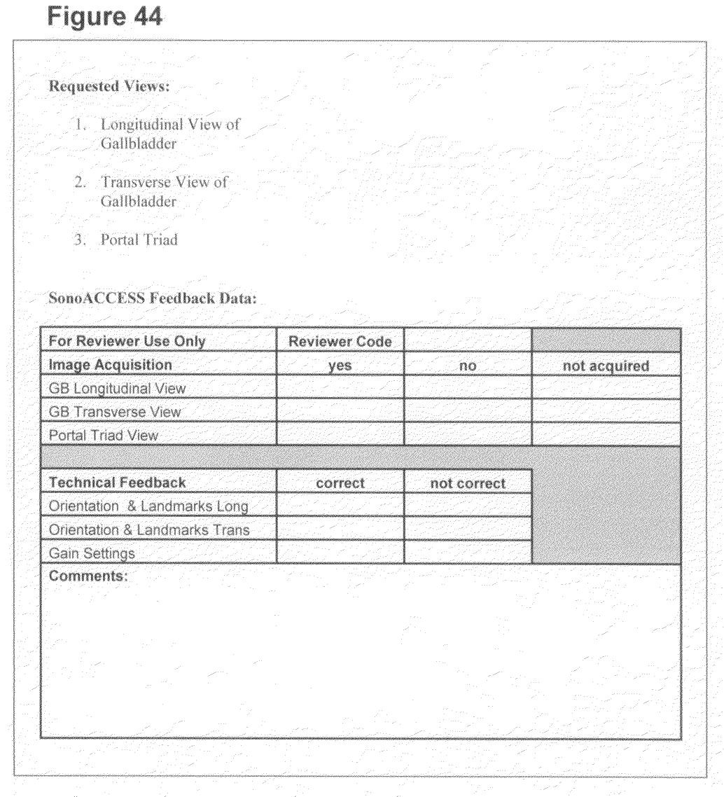

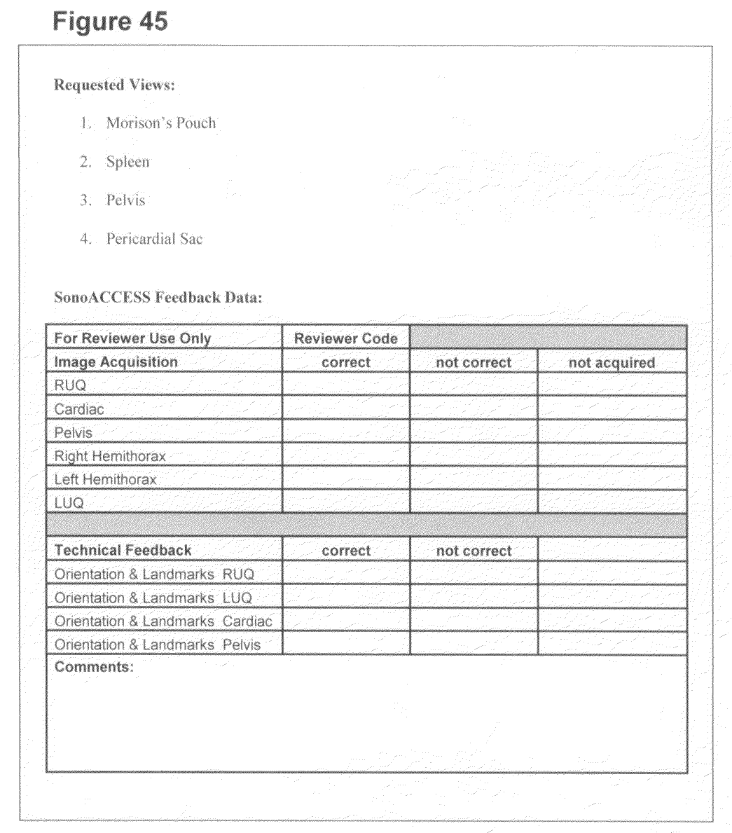

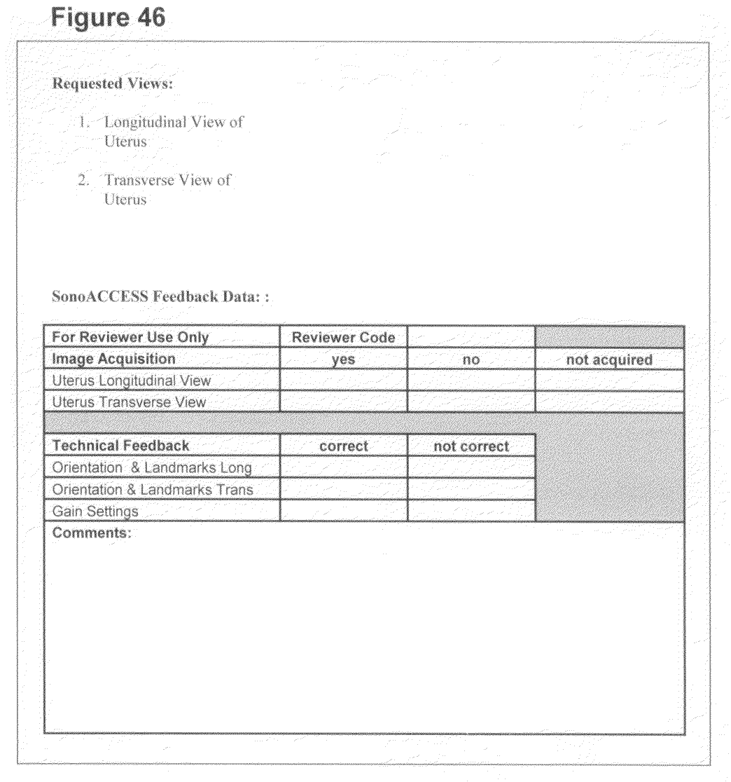

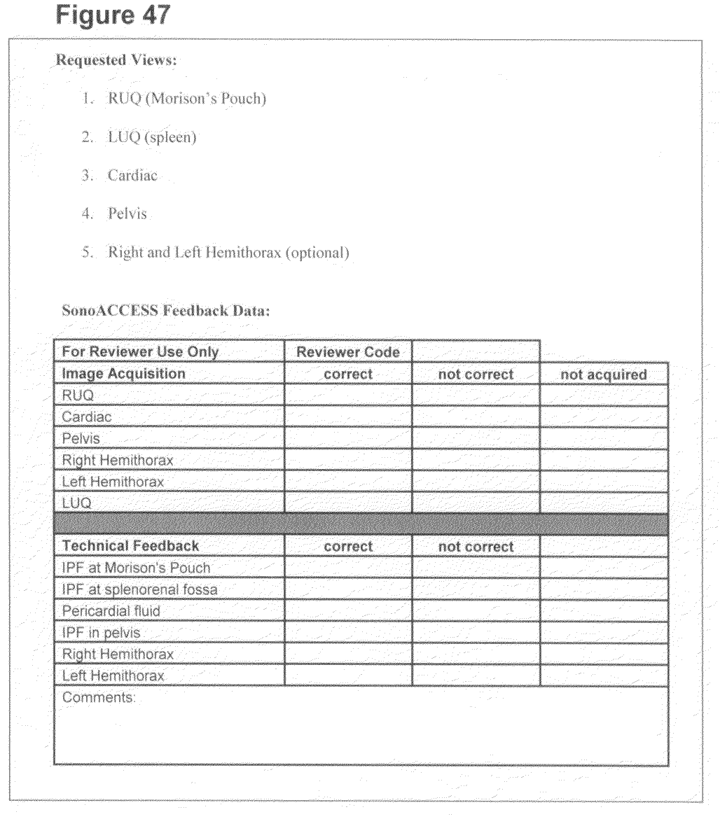

FIGS. 36-47 exemplarily shown options for the general layout and information requested for various standard templates (aorta, cardiac, biliary, trauma, OB/GYN, and other/trauma, respectively). FIGS. 36-41 show examples of the template form/view presented to the physician and FIGS. 42-47 show examples of the template form/view presented to the reviewer. Each template is designed for a focused examination prompting a physician to acquire specific image views and to answer a limited set of questions depending on the exam type (e.g., aorta, cardiac, OB-GYN, gall bladder, kidney, lung, trauma). The template provides for yes or no answers for key medical findings for 3-6 questions, for specific image views required (3-6 views), and/or for input as to the accuracy of the physician's clinical findings based on the images. It may allow the reviewer to provide feedback on whether the correct image views were acquired, the quality of the images, and whether the physician's findings are accurate based on the acquired images.

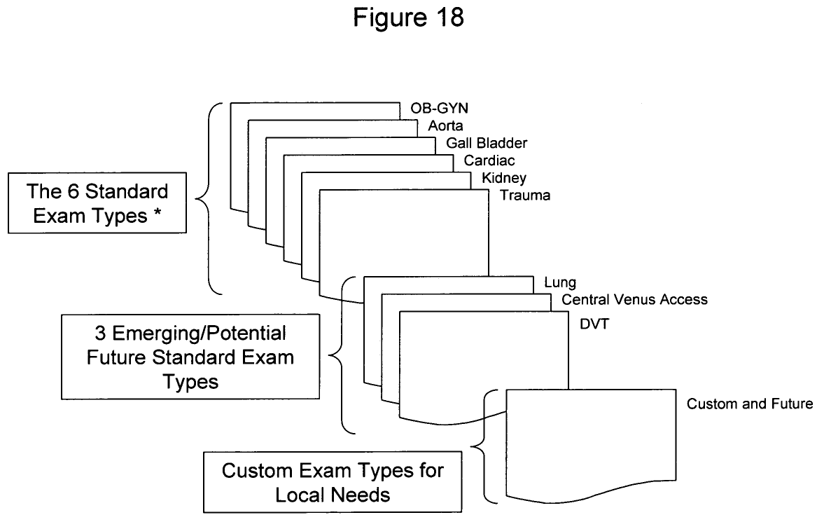

FIG. 18 shows types of templates for emergency physician use of mobile ultrasound and American College of Emergency Physicians (ACEP) guidelines. The 6 standard exam types shown (OB-GYN, Aorta, Gall Blader, Cardiac, Kidney, and Trauma) are from the guidelines published by the American College of Emergency Physicians. Over time or for specific localized reasons, new templates may need to be designed; therefore, emerging/potential future standard exam types (three illustrated here: Lung, Central Venus Access, and DVT) and custom exam types for local needs are also shown.

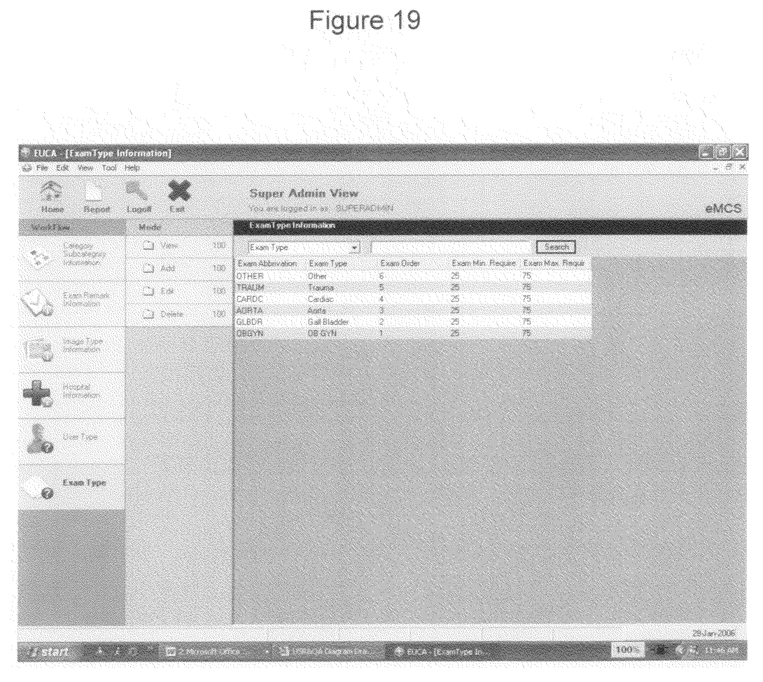

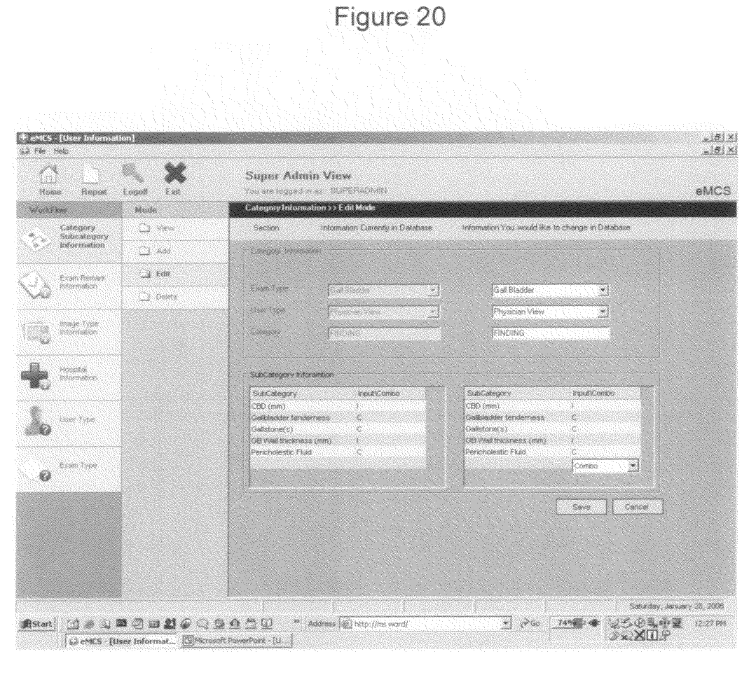

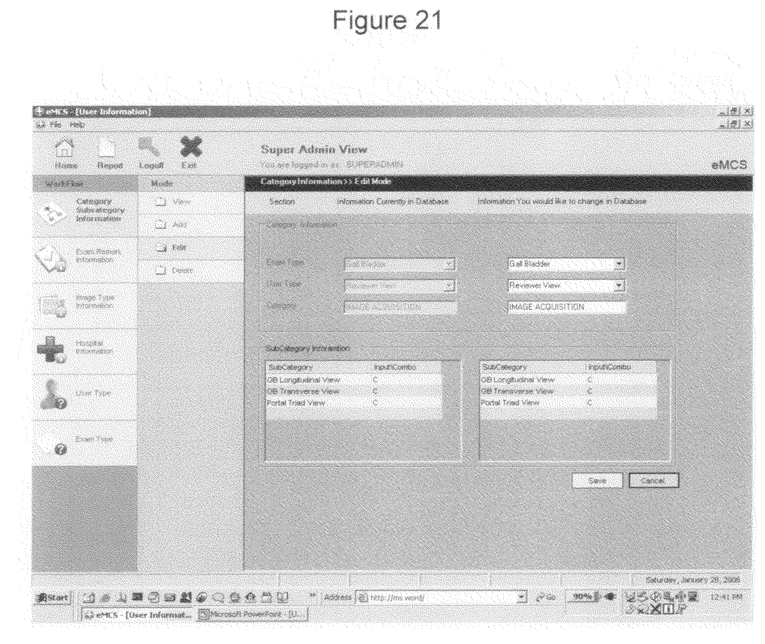

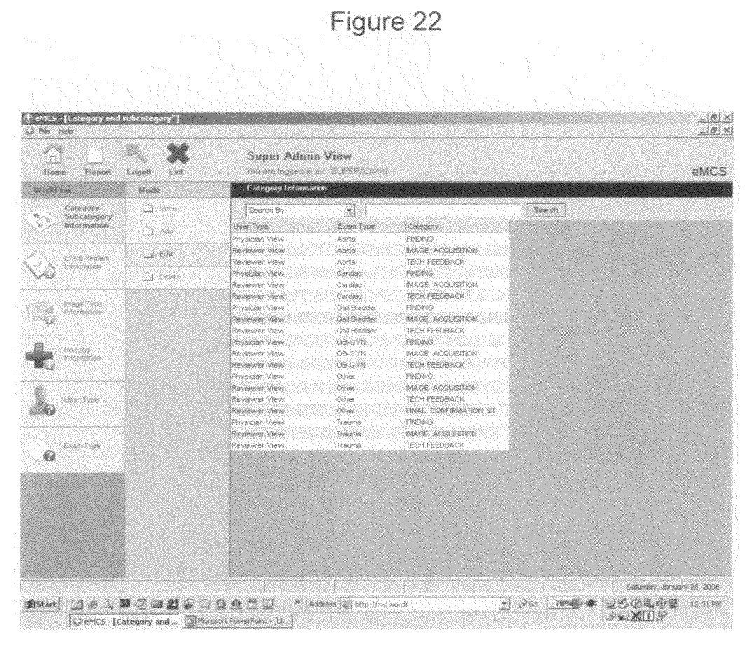

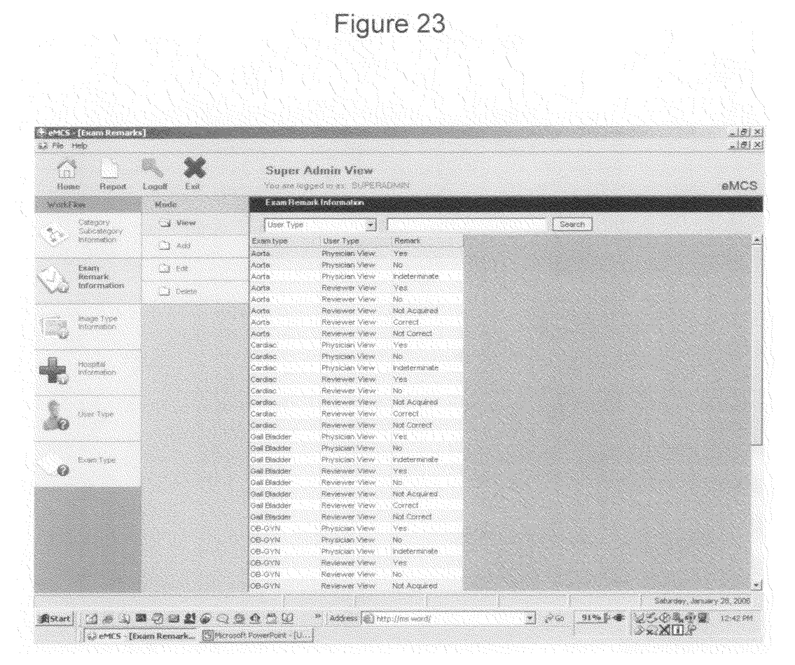

FIGS. 19 to 23 are screen views of the software according to present invention. These figures demonstrate the software providing for dynamically changing, enhancing and expanding the exam templates over time as credentialing requirements are enhanced and expand as new knowledge is acquired as to how to best use the various emerging portable medical image technologies for specific exam types.

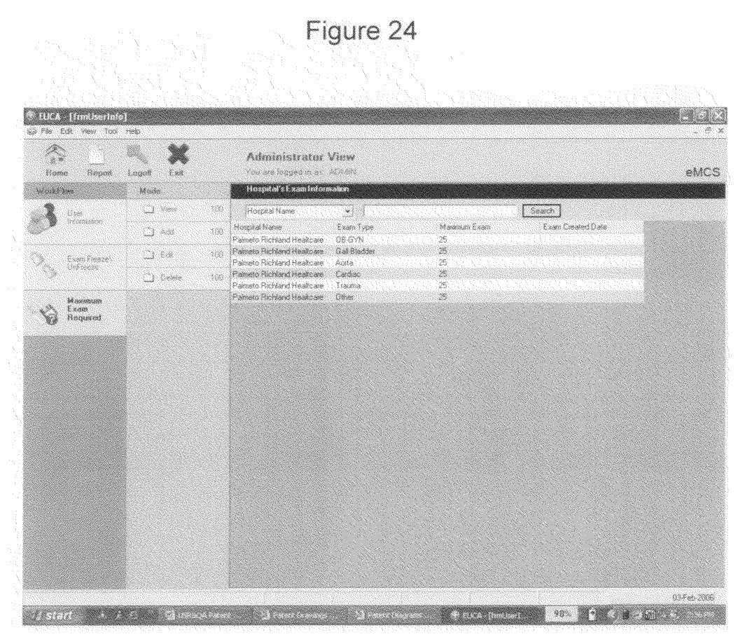

FIG. 24 shows how a specific institution can establish its own specific requirements for testing within ranges set by an organization such as the American College of Emergency Physicians (“ACEP”). These requirements include the number of exams needed for various exam types.

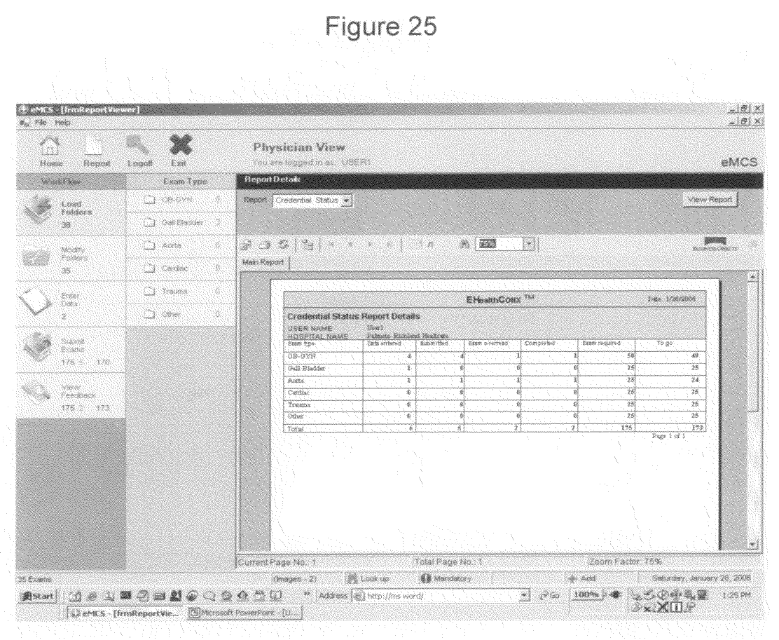

FIG. 25, illustrates how the present invention automatically tracks the physician's progress towards completing the credentialing requirements with a system generated report. Additional systems generated reports can be provided by the software according to the present invention, including but not limited to automated credentialing letters, complete scan profile reports, performance reports. A “system generated report” is any report that is generated automatically by the software automatically rather than requiring a person to manually compile the report. System reports can be presented either on the computer screen or can be printed as a paper report. Examples of reports include:
Physician Credentialing Status Report—show the status of the number of exams complete by exam type, how many required and how many “to go” to complete these requirements (see example below).

| | | | | | | |
|---|---|---|---|---|---|---|
| Exam | Exams | Technically | | Exams | Submitted | Feedback |
| Types | Performed | Limited | % | To Go | Pending | Ready |

OB-
GYN
Gall
Bladder
Aorta
Cardiac
Trauma

Kidney
Total

Group Credentialing Status Report—this report is almost identical to the Physician Credentialing Status Report but is compiled for groups of physicians to check on the overall status of a specified group of physician or the entire group of physician who are actively seeking to become credentialed.
Physician and Reviewer Activity History—this report presents transaction history including a list of exams that were submitted and reviewed for date ranges to be specified for both physician and reviewers. The report also can be sorted by physician, by reviewer, by date, exam type and by date.
Active and Inactive Physicians—this report presents a list of active and inactive physicians and can be sorted by department and by hospital.
Percentage of Completion—this report presents for active physicians their percentage of completion of the credentialing process and number and percentage of exams which were rejected for credentialing purposes.
Reviewer Activity Analysis—this reports present reviewer activity including the numbers of exams reviewed per a given period of time and comparative percentages of exams rejected by reviewer (i.e., hard graders versus easy graders).
Physician Watch List—this report presents a list of physicians who are in danger of not completing the process due to an unusually high number of exams which have been rejected and where remedial actions may be necessary.
Blank Credentialing Worksheets—the automated credentialing templates can be printed out as blank forms to be used in cases where a physician would prefer to use a manual form with a secretary or assistance entering the data into the system from the manual form
Completed Credentialing Worksheets—the completed templates can be printed out with the answers, feedback and underlying images included with the printout. These worksheets can be printed out either during the process or in total once the process is completed to be submitted to the physician's institution to provide evidence that the credentialing requirements have been me.
Hospital Credentialing Status Report—a report on an entire hospital status in credentialing all of its physicians including a list of all the physicians, percentages already credentialed versus working towards completion of credentialing requirements, specialty areas represented, locations, departments, etc.
Standard Letters and Memos—various standard letter and memos can be automatically printed by authorized reviewers or administrators to, for instance, document completion of credentialing requirements for a particular hospital, communicate to a physician that he is in danger of not completing the credentialing requirements until certain corrective actions are taken, etc.
System Activity Log—this report presents a record of who has accessed the system and database, for how long and how many exam transactions were processed both for physicians and reviewers. This report is for use by a systems administrator only.
Software Configuration Report—this report presents the software configuration including how many physicians are authorized to use the system to become credentialed, how many reviewers are authorized to provide feedback, the expiration date for the software, the software version, the exam types supported, the numbers of exams required for each exam type for that particular institution

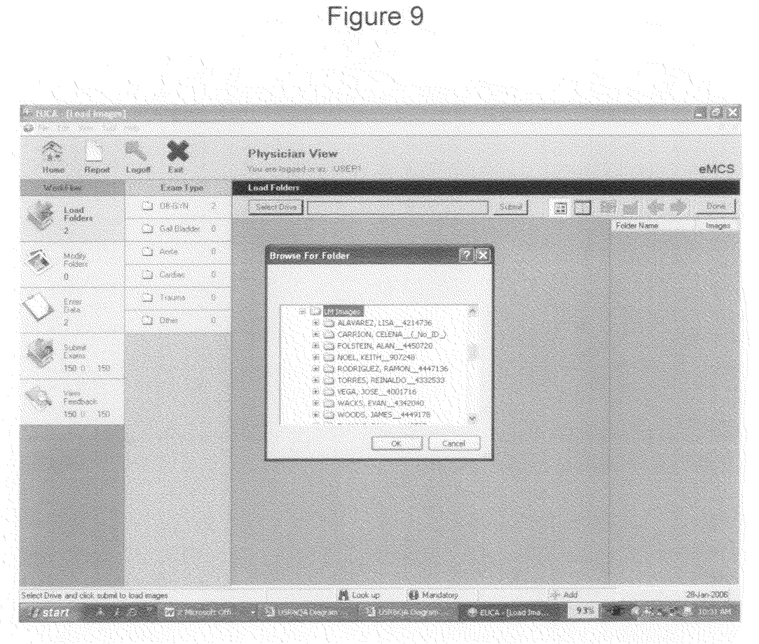

Referring briefly back to FIG. 8, Box 4 shows the physician (or an assistant) loading the images to the system from a storage device. Preferably, this is represented by the user screen pictured in FIG. 9, which shows the automatic load function as described above. The physician can direct the software to any standard directory, preferably Windows-based, where his or her images are stored and the software will automatically load individual images or image folders regardless of the type of images or the vendor equipment used.

As pictured in FIG. 9, the physician user can initially load image files that have been previously downloaded from an imaging device by pointing the software to a directory where these downloaded files exist and can be read. The downloaded files might reside on a floppy disk, a USB memory stick, a CD-ROM, or the hard drive of a personal computer depending upon how the download function of the particular vendor's equipment works. The system simply reads these folders from whatever directory on whatever storage device the physician points the system to as presented in FIG. 9. In FIG. 9, the user is pointing the system to the “LM Images” directory which contains a list of image folders.

A flowchart of a preferred process according to the present invention is presented in FIG. 8. The first three steps (boxes 1, 2 and 3) show how a physician uses a medical imaging device to perform an exam, to view and store digital images, and to transfer (or “download”) images from the device onto one of several different types of electronic storage medium (e.g., floppy disk, USB memory stick, hard drive in a computer). The process boxes in FIG. 8 marked with asterisks (*) are unique functions that can only be performed as part of an integrated system that facilitates a review and QA process.

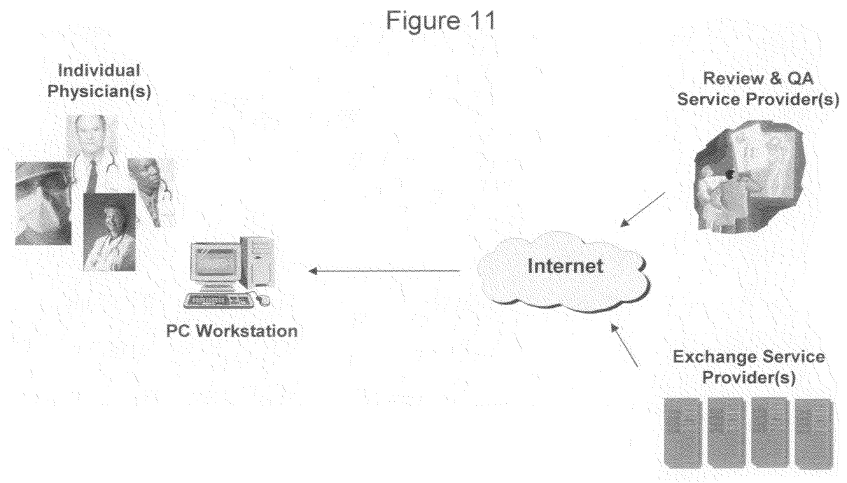

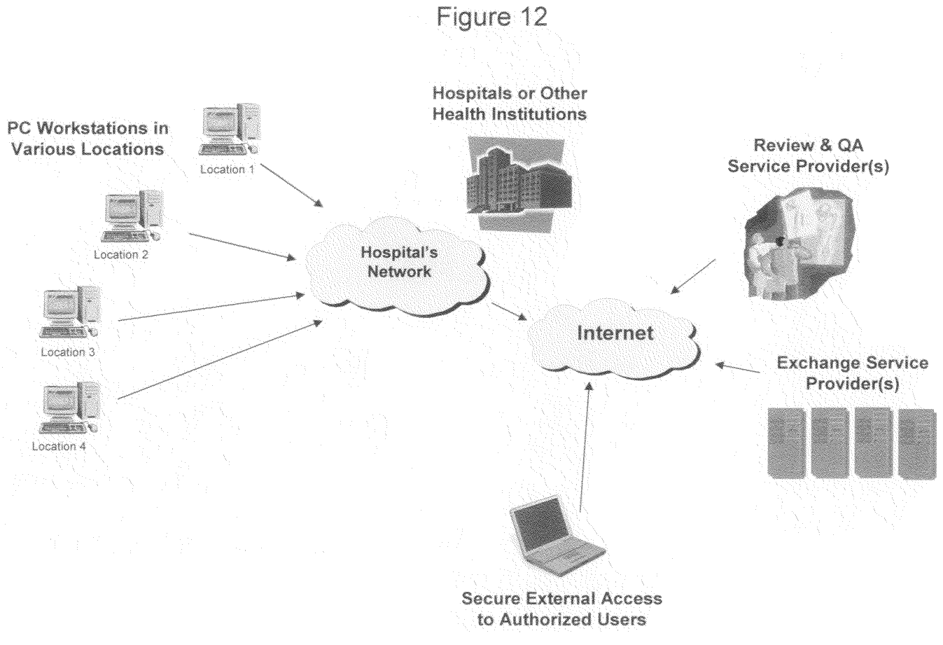

As shown in FIGS. 11 and 12, the software provides for Universal Encryption of not only image files initially loaded but also any additional data entered into the embodiment by the physician that are associated with a particular image or image folder. An exam folder may be encrypted for and prior to its transmission over the Internet. Additionally, as soon as an exam file is saved, it is encrypted. Whenever new data is appended to the file, the file may be re-encrypted and saved again. Encryption may be performed prior to the storing of the data even without the data being transmitted.

As shown in FIG. 11, the software provides for Universal Encryption for an individual physician. The PC workstation contains rich or “fat” client software that further protects data with the following components: physician specific views of templates, images, and/or exams; security by requiring user id and password supplied by service to login to the software; a unique serial number for software, encryption software, and a public key. In FIG. 11, the Exchange Service Provider(s) provide the following services: download or send CD with viewing software; issue user id and password; assign unique serial number for each instance of software; issue public keys; and de-encrypt exams for trusted reviewers.

As shown in FIG. 12, the software provides for Universal Encryption for multiple users in a hospital or health institution. The PC workstation contains rich or “fat” client software that further protects data with the following components: physician views; local user ID and password; single unique serial number for all instances of the software; embedded encryption software; embedded public key; and same software for both internal and external to the hospital's network. In FIG. 11, the Exchange Service Provider(s) provide the following services: provide viewing, review and administrative software; assign unique serial numbers for all instances of software; issue 1 public key; de-encrypt data when outside trusted reviewers are needed.

In FIG. 8, after box 5 (“Encrypt Images Stored”), the images and any data associated with them cannot be viewed by anyone other than the physician being credentialed, another authorized user, the original trusted source for the software, and reviewers and other users authorized by the trusted source to view selected image files and folders. A trusted source is a common term used to refer to any entity that issues encryption keys also known as digital certificates or digital security certificates to secure transmissions over the Internet of confidential data.

The preferred embodiment uses a double layer encryption approach (two levels of encryption). First, the embodiment encrypts the all images and data with a single encryption key issued with each different customer release of the software to either an individual physician or a group (e.g., a hospital). Second the software will only allow transmission of the image and date over the Internet using an additional secure HTTPS protocol involving double key encryption implemented using digital certificates issued by a trusted source (in the case of the preferred embodiment, this is issued by the Credentialing Service Provider).

Step 5 in FIG. 8 is “Encrypted image stored”. As soon as physician saves any image, it is encrypted using the physician's or group's key, uniquely issued by the service for each user or group of users of the software, and the image and data are stored in its encrypted form. Only the user or group of users using the same version of the software can decrypt the image and data stored by that version of the software to make it available for viewing. Different users or groups who have the same software but a different version (and software serial number) are not able to open data sets that they somehow received from other users of the same software but with a different software serial number. An additional protection is that users cannot access the software without a User ID and password.

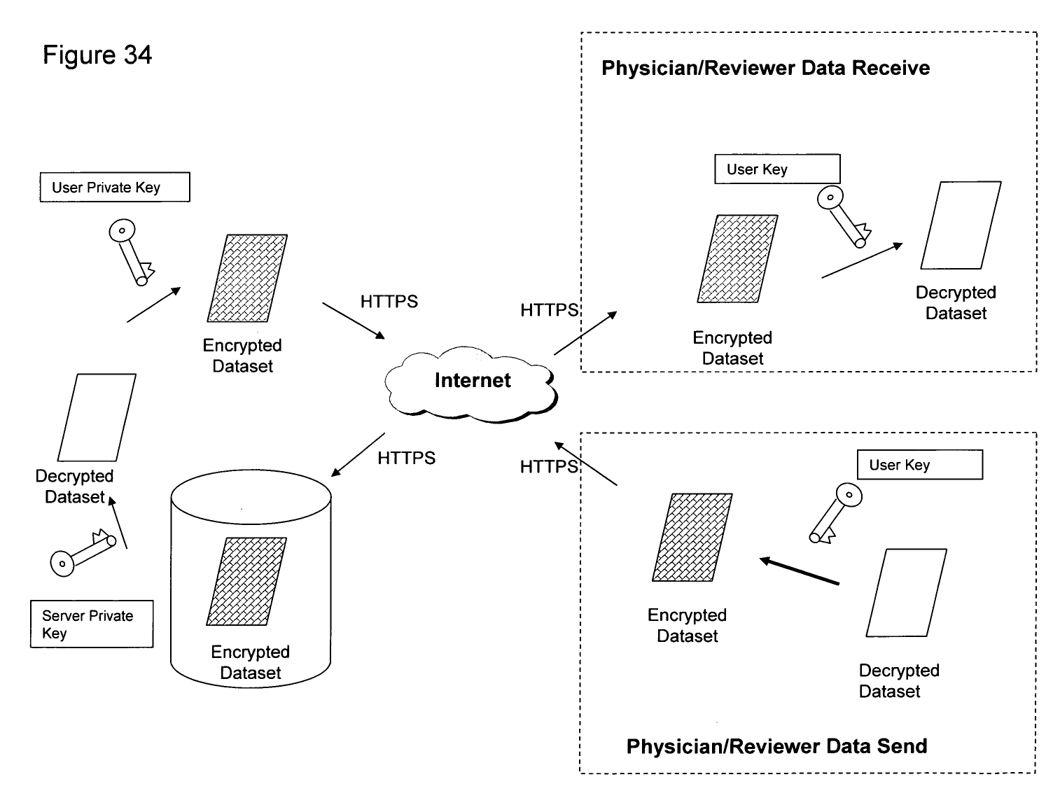

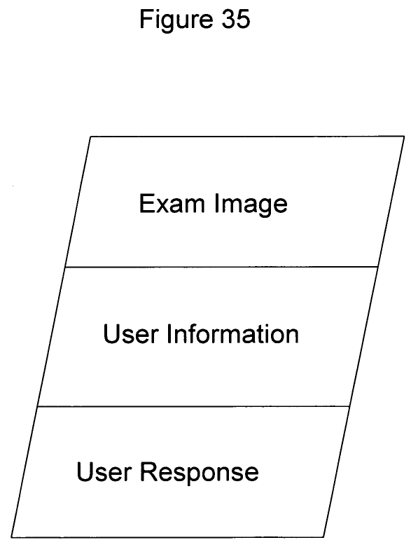

Additionally, the encrypted image and data is part of a data set that can be transferred over the Internet to the service provider. The dataset consists of the image, physician user information and the data entered into the embodiment data set by the physician (and subsequently with any Reviewer Feedback added). FIG. 34 illustrates the process of how the present invention encrypts the Dataset using the user key and HTTPS (thereby providing two layers of data encryption). FIG. 35 illustrates the exam folder Dataset which is an XML file.

The Dataset will be transferred to the service provider using HTTPS secure communications protocol. HTTPS or S-HTTP is a common protocol for transmitting data securely over the Internet. HTTPS is designed to transmit data securely using a public key and a private key with the public key provided by a trusted source using a digital certificate given over the Internet to a user making the transmission.

With HTTPS, the client workstation additionally encrypts the previously encrypted dataset (two layers of encryption) using a session key and the server will decrypt it using the Exchange Services private key. This will ensure that even if unauthorized persons receive the data during network transfer, they will have to break two layers of encryption to view the dataset.

The server stores the dataset in the encrypted form. But it has all unique user keys to decrypt the dataset when needed. Upon any authenticated reviewer's request, the encrypted Dataset will be decrypted by the server using the physician's unique encryption key and again encrypted using the reviewer's user key. The encrypted dataset will then be sent to the reviewer using the HTTPS protocol and decrypted by the reviewer's user key on the reviewer's computer.

The process is repeated when the reviewer returns his or her feedback so as to return the feedback to the physician who originally created the exam data set.

Both the initial software encryption and the HTTPS encryption using a public and private key approach are accomplished with a variety of well-known vendor products and open source software tools. For instance, encryption software and digital certificates for HTTPS and encryption of Internet transmissions are supported by firms including Verisign, Entrust, Network Solutions and Geo Trust. The software for basic single key encryption to encryption images and data stored locally by a single user or group of users is provided by firms such as PGP Corporation.

As pictured in FIG. 7, the preferred embodiment includes different “views” of the software for a physician, reviewers or “overreaders”, and administrators (including both a local administrator used for instance by a hospital group user and a broader “super administrator” used for example by the Exchange Service Provider). The software also includes several underlying functions used to integrate the different views into a single integrated system and method for performing image exam reviews and QA.

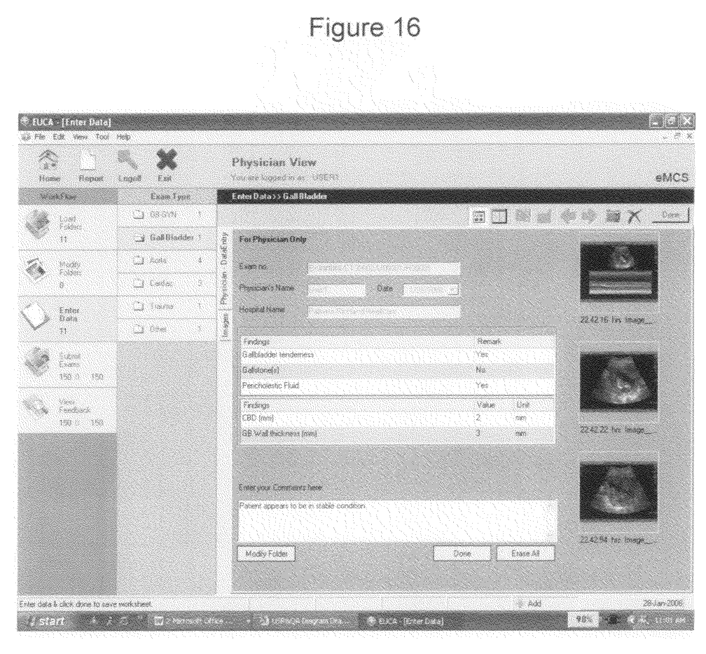

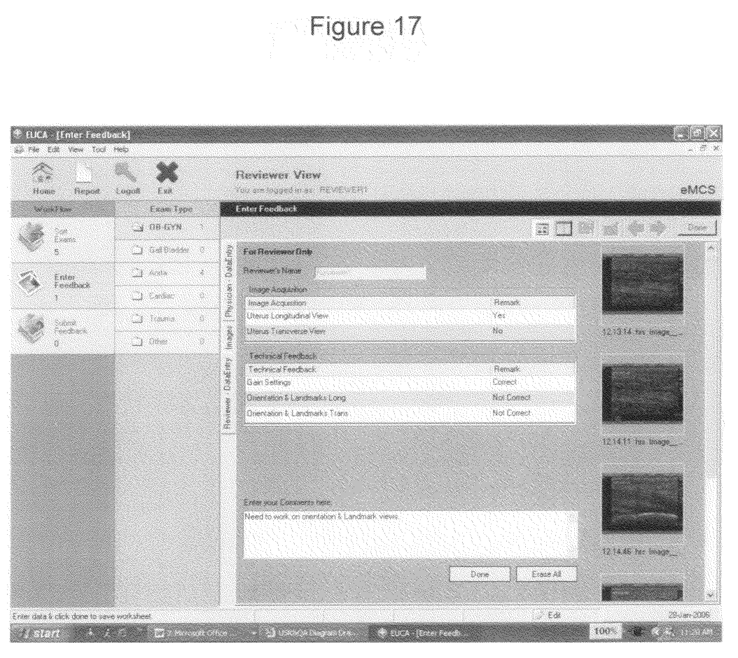

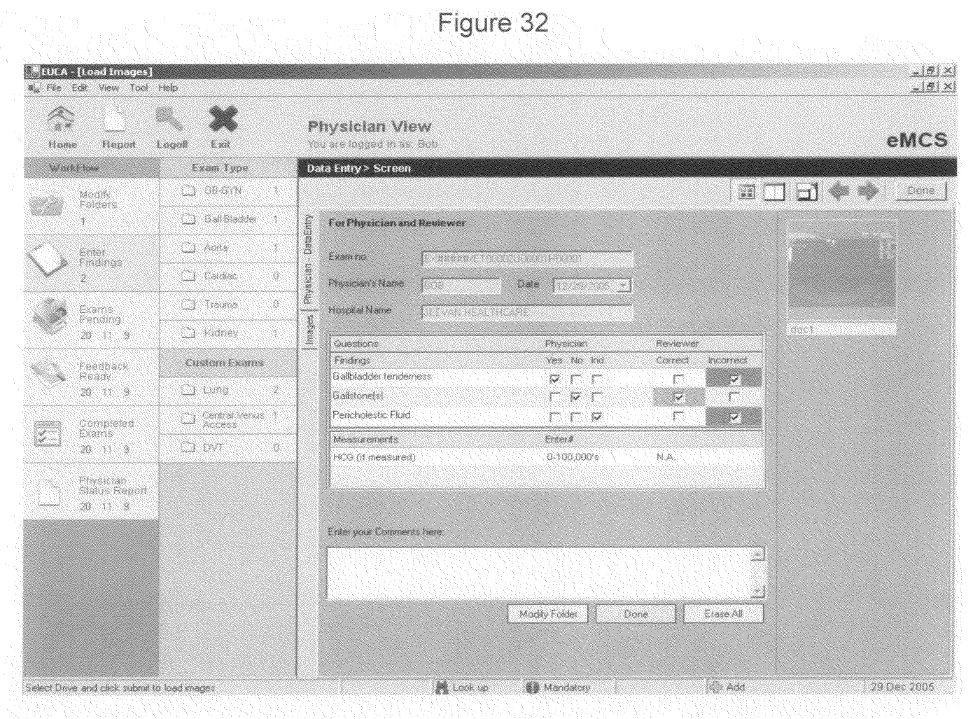

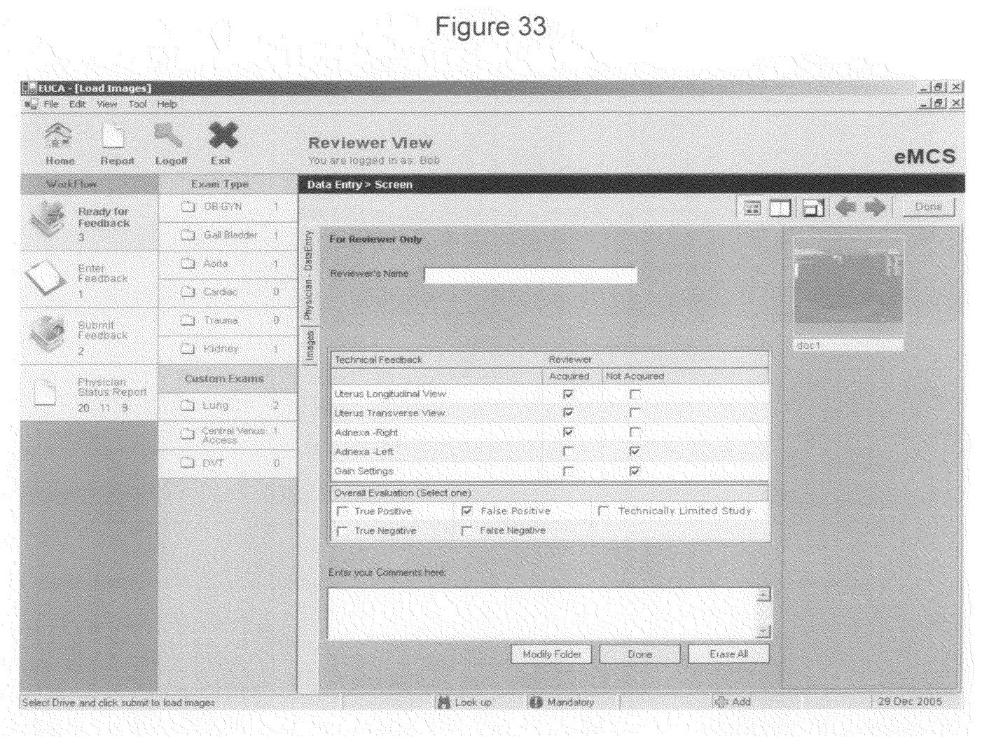

FIG. 16 shows the physician user screen demonstrating the appearance of an exemplary exam template. FIG. 32 shows another exemplary screen. Likewise, FIG. 17 shows an exemplary reviewer user screen illustrating an exemplary exam template. FIG. 33 shows another exemplary screen. Further, it will be appreciated that the screen of FIG. 32 may be output both in a physician view and a reviewer view. According to this exemplary embodiment, it may be that a user may interact with some of the fields of the screen exclusively in one of the views and with some other of the fields of the screen in the other view. For example, the fields appearing in the Physician column may be checked off in the physician view only, while fields appearing in the Reviewer column may be checked off in the reviewer view only, even though it may be that all of the data, including any check marks may be viewable in both views. Generally, the system provides a mechanism for physicians and reviewers to exchange exam templates (instances of exam templates) for examinations performed by physicians using medical imaging devices.

The images loaded from an ultrasound scanner or other medical imaging device may not be immediately suitable for ultrasound review and QA activities. As such, the preferred embodiment allows a physician to make slight modifications to image folders to prepare these folders for a review and/or QA process.

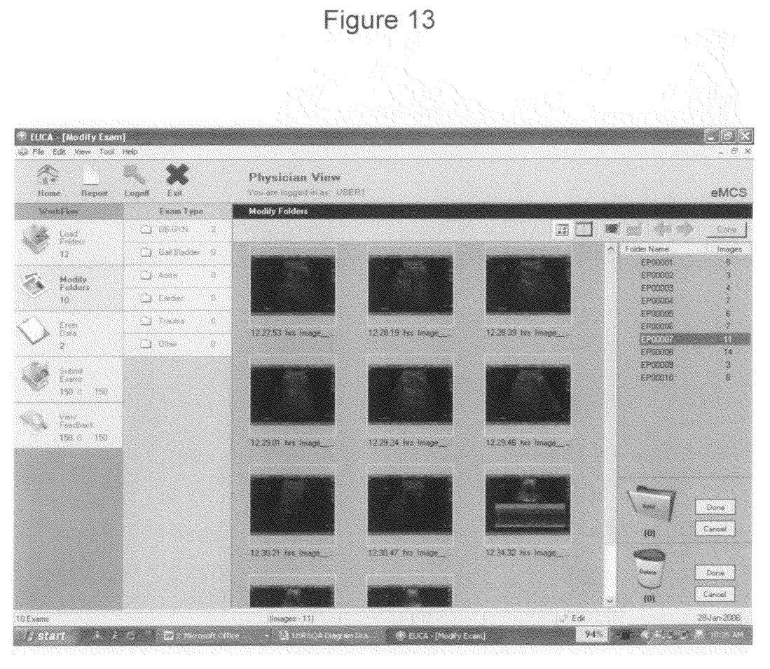

These preparation steps, if needed, are shown in flowchart boxes 6, 7 and 8 of FIG. 8. FIG. 13 illustrates these steps according to the present invention. The software preferably allows a physician to “split” image folders, to delete extra images included by the imaging device but not needed in the folder for the reviewer to provide feedback, and to assign individual folders to a specific exam type.

The ability to split image folders may be needed as medical image devices can be designed with an assumption that only one type of patient exam will be performed on a single patient in a single day (e.g., cardiac, aorta, gall bladder, lungs, etc.). But for training purposes, a physician may choose (with a patient's permission) to conduct multiple exam types on the same day. For instance, certain ultrasound devices will place all of images generated for the same patient on the same day into a single folder regardless of the type of exam conducted (e.g., OB-GYN, cardiac, gall bladder). Preferably, the split function of the present invention allows a physician to split up a folder into several folders by “dragging and dropping” individual images into the split folder icon (see lower right portion of FIG. 13). Additionally, a physician might acquire more images than needed or that are allowed by an outside review service for training and QA purposes. In that case, the software allows the user to drag and drop individual images to delete and then restructures the folders with the images deleted.

Finally, the software allows the physician to categorize folders by exam type. In FIG. 13, the six standard exam types proscribed under guidelines (such as the ACEP) for credentialing emergency physicians to use ultrasound are presented under the column “Exam Type”. The physician drags and drops the folders listed under folder name into the correct exam type category.

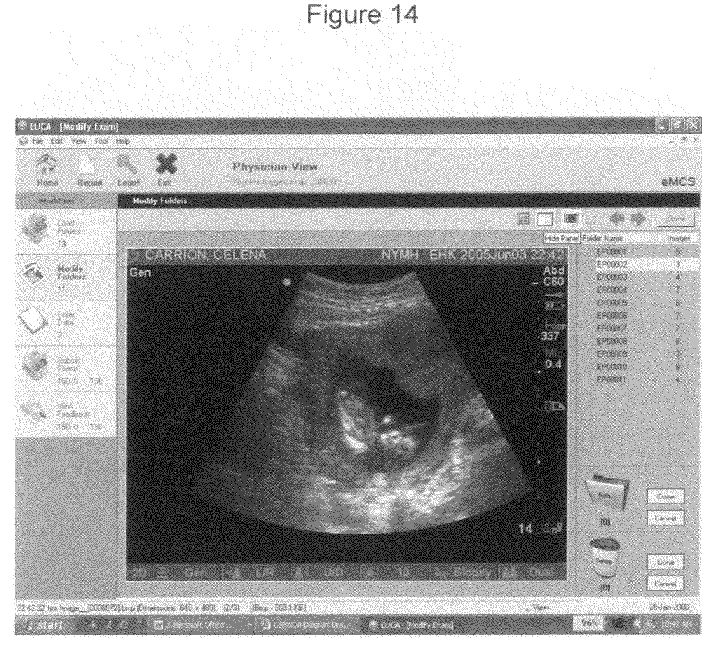

To further facilitate use of the images, the software allows the physician to manipulate the images for viewing. As an example, the physician can select any thumbnail of an image presented in FIG. 13 and enlarge the thumbnail for viewing as presented in FIG. 14. Additionally, the physician can manipulate the brightness and contrast of the images but cannot store these changes.

In boxes 4 to 11 in FIG. 8, the physician preferably assembles image exams to be reviewed including entering his or her medical findings from images acquired from the device. The system prepares and/or is used to prepare image exams for conveyance to a reviewer (e.g., encryption, splitting multi-exam folders, and deleting extra images, as explained above). In boxes 12 to 17 the reviewer provides the necessary feedback on the exam and returns the exam with the feedback to the physician. In Steps 18 to 22, the physician receives the exam with the feedback and acknowledges receiving the feedback.

In addition, a reviewer has the option to annotate images. This means that a reviewer has the ability to draw on the images by adding markings that look similar to the markings from a Magic Marker. Annotations can be used by a reviewer to create markings to an image for review by the physician such as circling a key feature of the image, drawing arrows or adding any other markings. Additionally, annotations can be used to either write or type text onto the images. All of these annotation features are intended to create additional avenues for the reviewer to effectively communicate his or her feedback back to the physician.

Additionally, the reviewer can manipulate the brightness and contrast of the images and store these changes as feedback. This illustrates to the physician that future improvements are required in the appearance of images that the physician directly captures with the imaging device.

In FIG. 7, message management is a method for ensuring that all exams that are sent for review are acknowledged. If an acknowledgement is not received the message is resent until it is successfully acknowledged by the receiving computer processor or node. Likewise, message management ensures that duplicates of the same exam are not inadvertently stored. Message management refers to software that implements a computer communications protocol that may require a network node receiving a message to acknowledge receipt by transmitting an acknowledgement message to the transmitting network node. The communication may be retransmitted until an acknowledgement message is received. Further, message management refers to software used to manage data transferred between computer processors and techniques for the receiving computer to send an acknowledgement back that an entire message has been received. This prevents problems when using communications such as wireless where part way through a communication the wireless connection is lost. In these cases, the receiving computer will know it has not received 100% of a discrete data transmission and the sending computer as a result will know to hold the transmission and attempt to send it again. This is basically two parallel inventory systems where both the sender and the receiver are keeping an inventory of what is being sent and received using common codes to measure if an entire data transmission has made it (e.g. hash totals), and discussing back and forth with each other whether a transmission has been successfully received or has to be resent with both processors agreeing in common what is successfully received and what has to be resent to avoid sending and storing duplicates.

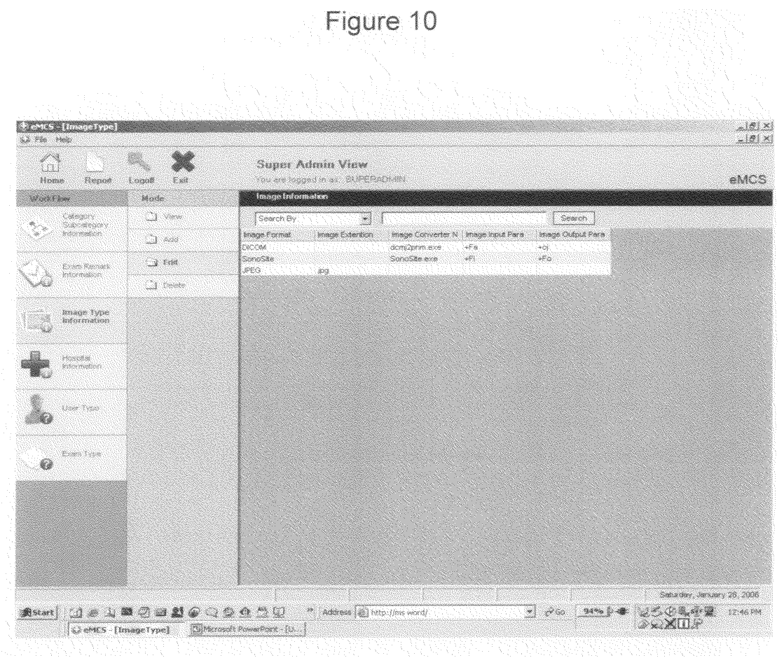

FIG. 10 presents the administrator's view of tables loaded with the different vendor's image file and folder formats. Many different formats can be loaded into this table to accommodate different vendor's file types and image folder formats. Preferably, as shown in FIG. 8, after the images are loaded (box 4), the software encrypts the images when storing them in a database (box 5).

As shown in FIG. 7, the system according to the present invention is preferably designed to securely exchange medical imaging exams over a network. A network is required for the transfer of information from one portion of the system to another. This network may be one or more of the following: the Internet, a single personal computer, a hospital virtual private network, integration with a hospital PACS system (Picture Archiving and Communication Systems), partial integration onto the medical imaging device, email transfer, network server, or integrated with an educational electronic content. Preferably, the network is the Internet. In cases where a non-email exchange is executed over the Internet, an Exchange Services Provider preferably facilitates this exchange.

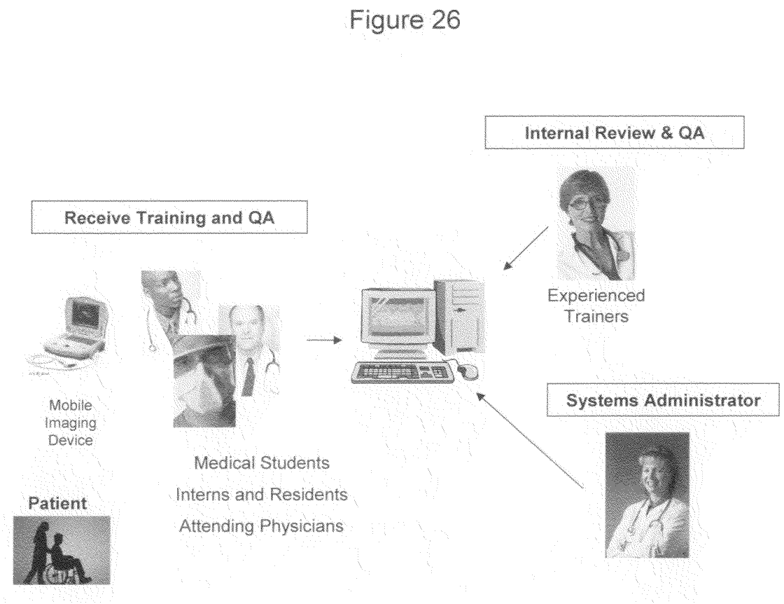

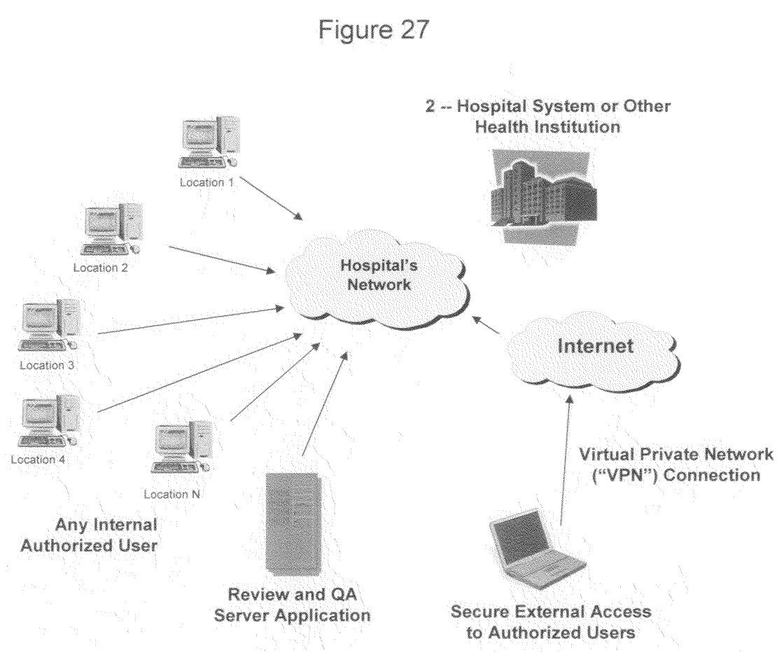

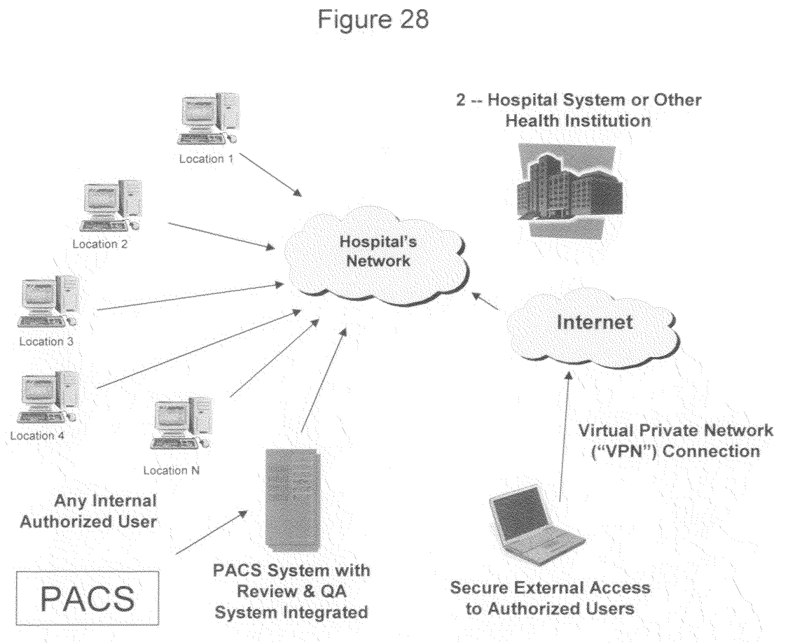

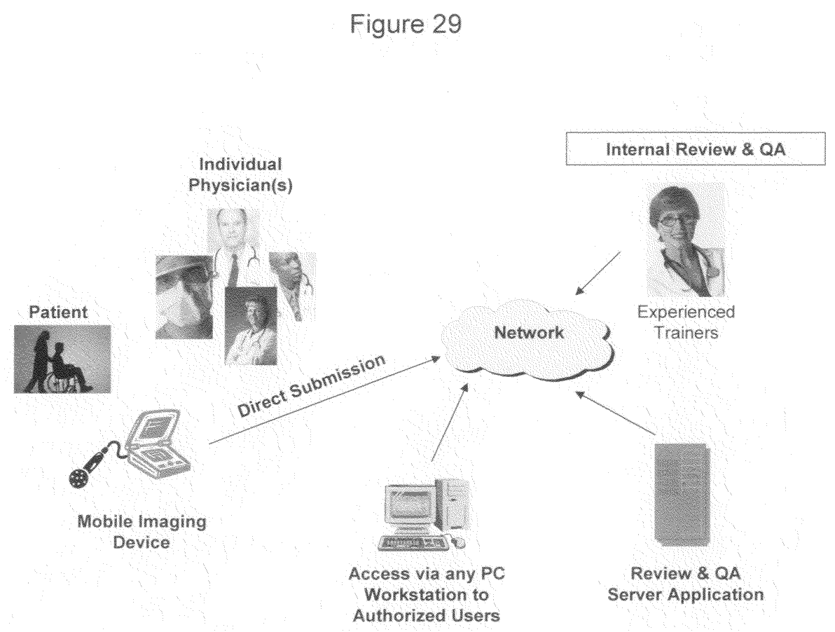

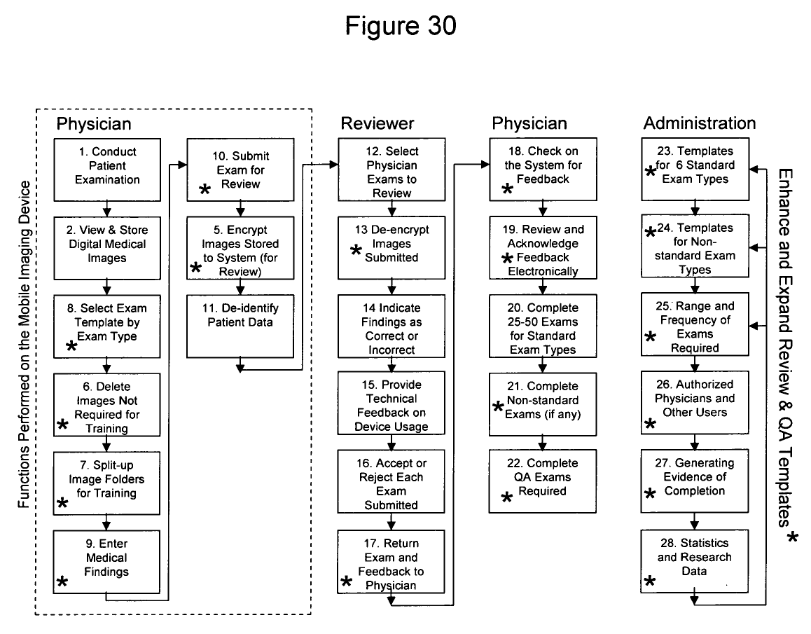

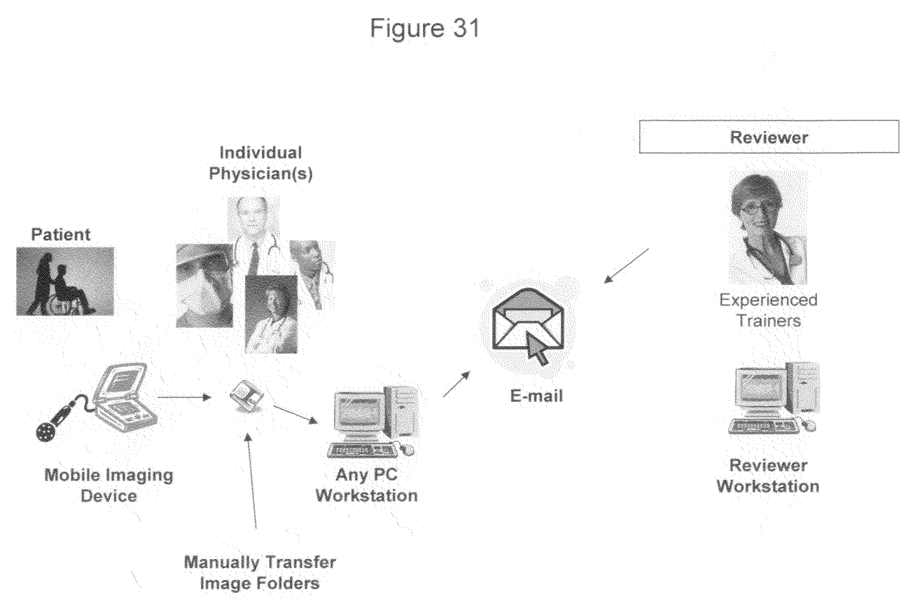

FIG. 26-31 present various alternative options for implementing the system according to the present invention. FIG. 26 is a Single Department PC Application. FIG. 27 is a Hospital Virtual Private Network (“VPN”). FIG. 28 shows the system integrated with a PACS. FIG. 29 shows the system partially integrated onto the Medical Imaging Device. FIG. 31 shows E-Mail Transferring the Exam Templates. Each is described below. FIG. 30 presents the workflow for the alternative embodiment of the present invention shown in FIG. 29.

FIG. 26 shows the system according to the present invention operating on a single personal computer (“PC”) workstation. For instance, this workstation may be in an emergency department. In this case, both the physicians and reviewers would each need to work in the same department and use the same single PC workstation (but preferably not at the same time). This configuration would be particularly suitable for departments that support medical schools and/or residency programs where it is relatively easy to provide feedback internally. Another advantage is that the system can be managed without having to use an outside Exchange Service or to secure the services of outside reviewers.

The disadvantage is that multiple users will all have to use the same single PC workstation. However, given this embodiment is mostly for educational and QA purposes, the volume of activity will be much lower than otherwise might be the case for other types of high volume transaction systems (e.g., accounting systems). As such, a single PC workstation installation, in some instances, is expected to be sufficient for teaching institutions that would otherwise have no system or would be using manual forms and/or video tapes.

FIG. 27 presents another alternative embodiment where the system of the present invention is operated over an internal network (i.e., “intranet”) in a fashion that is similar but not identical to how the system would work over the Internet. In this case, physicians in multiple locations within a single health system could use the system to collect ultrasound images and other data in multiple locations within a health system and transfer this exam information, including findings and interpretations, to reviewers in different locations throughout that particular institution (but not necessarily outside the institution given the particular security constraints of the health institution).

Parts of the system can also operate either separately from the ultrasound device or it can be an integral part of the ultrasound device. For instance, many ultrasound devices today are manufactured using standard computer components including use of commonly used operating systems processors such as Microsoft Windows, Linux or VxWorks. The ultrasound systems itself can be used to process the system with the physician documenting findings and interpretations directly on the ultrasound machine. Then the system directly transfers the resulting exam images and other information to the database for reviewer access. The transfer of ultrasound exams can be performed using whatever mechanisms are supported by the vendor's ultrasound device including Ethernet connections, docking stations and wireless connections.

In FIG. 30, the initial Physician component of the invention runs on the imaging device itself (see dotted line in FIG. 30). This may be more efficient than the process shown in FIG. 8 because the physician is not required to download and (re)load images from a medical imaging device. In this alternative embodiment, rather than downloading images from a medical imaging device, the physician can assemble and enter findings for an exam to be submitted for review on the medical imaging device directly. The physician can submit the exams directly from the imaging device including potentially submitting exams directly from the device to the Internet for reviews.

The advantage of this alternative embodiment, shown in FIG. 30, is efficiency. Two steps from FIG. 8 are eliminated (i.e., box 3, Download Digital Images to a Storage Device and box 4, Load Images to the System). However, this embodiment requires coordination and cooperation of imaging vendors to integrate a portion of the invention onto their devices in order for this alternative embodiment to function as presented in FIG. 30. The process boxes in FIG. 30 marked with asterisks (*) are unique functions that can only be performed as part of an integrated system that facilitates a review and QA process.

Certain modifications and improvements will occur to those skilled in the art upon a reading of the foregoing description. By way of example, the imaging device of the present invention could be substituted with other portable diagnostic equipment, as these may also require trained and certified physicians. It will be appreciated that the present invention can be implemented in a variety of forms and may be applied to the credentialing of a user to operate any device; not only a diagnostic device. Therefore, the true scope of the embodiments of the invention should not be limited to the particular examples with respect to which embodiments are described above. Also, the present invention can be adapted for any clinical based medical specialty. Further, the present invention can be modified for use as a post credentialing QA system.

Also, video clips (i.e., stored records of medical images recorded over time that can be used to present motion inside the body) could be substituted for still pictures of medical images. Further, video streaming could be substituted for either still images or video clips and on-line templates substituted for batch submissions of templates so that a reviewer can provide real-time or near real-time credentialing feedback to physicians as they are using imaging equipment in a remote location. The system could also be modified for non-credentialing and QA purposes for medical schools to train medical students who do not yet need to meet any specific credentialing requirements but could nonetheless benefit from a version of the system customized for pure learning purposes. Finally, a “thin” client approach could be substituted for a “fat” client approach assuming that sufficient security controls can be put in place within a hospital or other group of users who are otherwise using the system for internal purposes. In this case, the secure HTTPS protocol would not be replaced for Internet transmissions but the single key encryption for local images and data stored would not be utilized or would need to be implemented through some other means other than a “fat” client application. All modifications and improvements have been deleted herein for the sake of conciseness and readability but are properly within the scope of the following claims.

What is claimed is:

1. A system for evaluating a medical imaging device user comprising:
a. at least one interactive electronic template for evaluating a medical imaging device user;
b. a storage medium for storing at least one image created by the medical imaging device user with a medical imaging device, wherein the at least one image correlates to the at least one interactive electronic template;
c. software running on a computer operable to receive and store the at least one image, wherein on a display having a user interface and the at least one interactive template provided by the software, a reviewer can review the at least one image when the storage medium is accessible on the computer.

2. The system of claim 1 wherein the at least one interactive electronic template tests the medical imaging device user's use of the medical imaging device and/or tests the medical imaging device user's interpretation of the at least one image taken with the medical imaging device.

3. The system of claim 1 wherein the at least one image is created during a medical examination of a patient.

4. The system of claim 3 wherein the at least one interactive template comprises at least one prompt relating to a focused examination.

5. The system of claim 4 wherein the focused examination is an aorta, cardiac, gall bladder, kidney, lung, trauma, or obstetrics-gynecology examination.

6. The system of claim 1 wherein the medical imaging device uses X-ray, computer tomography, magnetic resonance imaging, ultrasound, computer radiography, mammography, and/or nuclear medicine technology.

7. The system of claim 1 wherein the medical imaging device is a handheld medical imaging device.

8. The system of claim 1 wherein the at least one image is at least one video clip.

9. The system of claim 1 wherein the software combines the at least one image with the at least one exam template to create at least one exam, wherein the software stores the at least one exam on the computer, and wherein the software submits the at least one exam to a reviewer for evaluation.

10. The system of claim 1 wherein the at least one image is at least one image taken of a patient, further comprising patient information associated with the at least one image, wherein the software protects the patient information by de-identifying the patient information from the at least one image.

11. The system of claim 1 wherein the software is operable to present a first version of the at least one interactive electronic template to the medical imaging device user and a second version of the interactive electronic template to the reviewer.

12. The system of claim 1 wherein the computer is accessible via a network that includes at least one computer and the medical imaging device.

13. The system of claim 12 wherein the software is operable to allow the reviewer to provide feedback to the medical imaging device user while the medical imaging device user is using the medical imaging device.

14. The system of claim 13 wherein the at least one exam template comprises prompts to the medical imaging device user for key medical findings and specific image views.

15. The system of claim 14 wherein the software is operable to provide an interactive user interface wherein the reviewer can evaluate the accuracy of the key medical findings based on the specific image views, determine whether the specific image views were correctly acquired, evaluate the quality of the at least one image, determine whether the medical imaging device user performed acceptably.

16. A method for evaluating an medical imaging device user comprising the following steps:
a. providing at least one interactive electronic template;
b. a medical imaging device user conducting a patient examination with a medical imaging device;
c. creating at least one image with the medical imaging device, wherein the at least one image correlates to the at least one interactive electronic template;
d. transferring the at least one image to a storage medium;
e. the medical imaging device user entering at least one finding prompted by the at least one interactive electronic template and relating to the at least one image, thereby forming at least one exam comprising the at least one image, the at least one exam template, and the at least one finding;
f. the medical imaging device user electronically submitting the at least one exam for review; and
g. the reviewer evaluating the at least one exam, thereby evaluating the medical imaging device user.

17. A method for evaluating at least one medical imaging device-generated image comprising the following steps:
providing software running on a computer, wherein the software is operable to perform the following steps:
a. receiving at least one medical imaging device-generated image created by a medical imaging device user from a medical imaging device;
b. storing the at least one medical imaging device-generated image as corresponding stored image(s) on the computer;
c. providing a user interface on a display in data communication with the computer, wherein the user interface comprises an interactive template and the stored image(s);
d. receiving inputs from a reviewer via the interactive template relating to the stored image(s), thereby evaluating the at least one medical imaging device-generated image.

18. The method of claim 17 wherein the user interface is operable to assign each stored image to a specific interactive template.

19. The method of claim 17 further comprising the step of selectively activating template options for evaluating the medical imaging device images, wherein the template options comprise comparing specific images, deleting specific images, annotating specific images, and de-identifying patient information.

20. The method of claim 17 wherein the medical imaging device and the computer are interconnected via a network and the interactive template further comprises a prompt for the medical imaging device user regarding a medical examination and corresponding creation of the medical imaging device-generated image.
